(19) United States
(12) Patent Application Publication
(10) Pub. No.: US 2006/0088902 A1
(43) Pub. Date: Apr. 27, 2006

(54) Methods and materials for the identification of antifungal substrates in filamentous fungi

(76) Inventors: Cornelis A.M.J.J Van Den Hondel, GOUDA (NL); Arthur F.J Ram, TB Hillegom (NL); Robbert D Damveld, RA (NL); Mark Arentshorst, Noordwijk (NL)

Correspondence Address: KALOW & SPRINGUT LLP, 488 MADISON AVENUE, 19TH FLOOR, NEW YORK, NY, 10022, US

(21) Appl. No.: 10/488,015

(22) PCT Filed: Aug. 28, 2002
(86) PCT No.: PCT/EP02/09639

§ 371 Date: Jun. 9, 2005

(30) Foreign Application Priority Data
Aug. 28, 2001 (EP) 01203423.7

(51) Int. Cl. C12Q 1/18; C07H 21/04; C12P 21/06; C12N 9/10; C12N 15/74; C12N 1/16

(52) U.S. Cl. 435/32; 435/69.1; 435/254.1; 435/193; 435/484; 536/23.2

(57) ABSTRACT

The present invention relates to a method for the identification of antifungal substrates, in particular substrates which are capable of combating filamentous fungi by disturbing cell wall biogenesis. A nucleotide sequence encoding α-1,3-glucan synthase including its promoter sequences is provided, obtainable from Aspergillus niger, which can be used to develop a reporter system for the identification of a new antifungal compounds in filamentous fungi. The invention relates also to antifungal substrates so obtained and to methods for applying such substrates, in particular in the treatment or prophylaxis of human and animal fungal infections, plant diseases caused by fungi or in the preservation of food against deterioration by fungal growth.

### FIELD OF THE INVENTION

The present invention relates to a method for the identification of antifungal substrates, in particular substrates which are capable of combating filamentous fungi by disturbing cell wall biogenesis. The invention relates also to antifungal substrates so obtained and to methods for applying such substrates, in particular in the treatment or prophylaxis of human and animal fungal infections, plant diseases caused by fungi or in the preservation of food against deterioration by fungal growth.

### BACKGROUND OF THE INVENTION

Filamentous fungi are important pathogens of plants and animals and form a serious problem in the food industry by causing food spoilage. A variety of compounds is known to have fungistatic properties and can therefore be used to inhibit the growth of fungi in a product. However, many fungistatic compounds are generally not accepted in food products or they have a non-natural image.

To avoid or fight fungal infections, an extracellular target such as the cell wall is highly attractive. However, filamentous fungi can adapt to the presence of cell wall lytic enzymes or inhibitors of cell wall biosynthesis by changing their cell wall architecture, thereby making the fungus less sensitive or even resistant to antifungal agents.

WO 97/16973 (Unilever) discloses compositions suitable for combating fungi in food and other products such as personal care products, comprising the combination of a fungal cell wall lytic enzyme and a natural microbial membrane affecting substance (MMAS), in an effective concentration. The preferred cell wall lytic enzymes are chitinase, β-(1-3)-glucanase and β-(1-6)-glucanase. The MMAS is exemplified by nisin, amphiphilic alpha-helix forming peptides, such as histatins and the polypeptide FASLLGKALKALAFQ, and fungal inhibitors, such as carvacrol and sorbic acid.

EP-A-1023836 (Unilever) discloses compositions inhibiting the outgrowth of fungi which are more heat-stable and comprising a first ingredient which inhibits the biogenesis of a normal fungal cell and a second ingredient which is capable of preturbing the structure of the cellular membrane of said fungi, so that either the cellular integrity is essentially lost and/or cell division cannot take place. The first ingredient preferably is a β-(1,6)-glucose polysaccharide or a branched polysaccharide having a β-(1,6)-glucose polysaccharide backbone, especially β-gentobiose and postulan fragments. The second ingredient suitably is a MMAS, preferably nisin, amphiphilic α-helix forming peptides such as MB-21, and fungal inhibitors present in herbs suitable for food preparation, such as carvacrol and sorbic acid.

There is still a need for further investigation of the restoration mechanism or rather the cell wall remodeling mechanism(s) of filamentous fungi after exposure to cell wall lytic enzymes or inhibitors of cell wall biosynthesis in order to be able to develop or identify new antifungal compounds with improved properties.

It is therefore an object of the invention to identify antifungal compounds which are either directly related to cell wall biosynthesis, cell wall remodeling mechanisms or to signal transduction pathway(s) involved that mediate(s) the cell wall remodeling mechanism.

### SUMMARY OF THE INVENTION

The present invention relates to a method for the identification of antifungal substrates, in particular substrates which are capable of combating filamentous fungi by disturbing cell wall biogenesis.

In an aspect of the invention a nucleotide sequence encoding α-1,3-glucan synthase (agsA) or Glutamine:fructose-6-phosphate aminotransferase (gfaA) from A. niger including its promoter sequences is provided, obtainable from Aspergillus niger, which can be used to develop a reporter system for the identification of new antifungal compounds in filamentous fungi.

In another aspect of the invention the use of said promoter sequence is provided for developing a reporter system for the identification of new antifungal substances which exhibit fungistatic or fungicidal activity against filamentous fungi.

In still another aspect of the invention antifungal compositions are provided comprising at least one compound which has been identified by the method according to the present invention in conjunction with a suitable carrier. Such compositions may also contain other active ingredients. The compositions are useful in a variety of applications, for example, in the treatment or prophylaxis of human and animal fungal infections, plant diseases caused by fungi or in the preservation of food against deterioration by fungal growth.

In yet another aspect of the invention new potential targets for the development of antifungal drugs are provided. It was found that expression of the obtained nucleotide sequence encoding Glutamine:fructose-6-phosphate aminotransferase and α-1,3-glucan synthase, respectively, and the development of in vitro assays allow the screening and identification of substances that interfere with the enzyme activities mentioned above.

In a further aspect of the invention a method is provided of selecting and identifying sequences which are useful for the screening of potential new fungicides, comprising the following steps:
1) identifying genes involved in the architectural changes in the fungal cell wall such as genes encoding cell wall proteins and cell wall biosynthetic enzymes;
2) developing one or more suitable reporter constructs using promoter sequences of one or more induced genes to monitor the response of the cell to stress conditions;
3) identifying regulatory elements from said promoter sequence involved in the regulation; and
4) evaluating the use of the reporter constructs for the screening of antifungal compounds in other fungi.

In yet another aspect of the invention a method is provided for the identification of new antifungal targets and the development of new reporter constructs for the identification of antifungal substrates in filamentous fungi, comprising the following steps:
1) identifying genes involved in the architectural changes in the fungal cell wall such as genes encoding cell wall proteins and cell wall biosynthetic enzymes;
2) using the enzymatic activity of proteins encoded by said genes that are induced upon cell wall stress, which proteins contribute to cell wall biosynthesis and cell wall remodeling, as potential targets for new antifungals.
3) evaluating the usefulness of identified genes encoding enzymatic activities involved in cell wall biosynthesis/remodeling, as an antifungal target.

The invention relates also to antifungal substrates so obtained and to methods for applying such substrates, in particular in the treatment or prophylaxis of human and animal fungal infections, plant diseases caused by fungi or in the preservation of food against deterioration by fungal growth.

These and other aspects will be further explained in the detalied description and examples below.

### BRIEF DESCRIPTION OF THE DRAWINGS

FIG. 1: Sequence alignment of a part of the four different Ags genes from Schizosaccharomyces pombe. Several primers for the isolation of a1,3-glucan synthase genes from A. niger and Penicillium crysogenum are indicated (see also Table 1).

FIG. 2: The four members of the Ags protein family identified in A. niger.

FIG. 3: Effect of CFW addition on growth of germlings. Germinated spores of A. niger were treated with increasing concentrations of CFW. Both the extend of hyphal tip swelling and the time required to resume growth is dependent on the concentration of CFW.

FIG. 4: Northern blot analysis of RNA isolated from CFW stressed and unstressed germlings. agsA; gfaA; fksA; and control loading.

FIG. 5: Schematic representation of Plasmid PagsA-AmdS.

FIG. 6: Analysis of PagsA-AmdS transformants A) Schematic representation of the pyrG locus after single integration of the PagsA-AmdS at pyrG. B) Southern blot analysis and ability to grow on aceetamide plates.

FIG. 7: Schematic representation of Plasmid PagsA-GUS.

FIG. 8: Analysis of transformants PagsA-GUS. A) Schematic representation of the pyrG locus after single integration of the PagsA-GUS at pyrG. B) Southern blot analysis and a Gus-activity assay on transformants containing a single copy integration of the pAgsA-AmdS-RC-pyrG- construct at the pyrG locus.

FIG. 9: Comparison of deduced amino acid sequences of partial α1,3-glucan syntheses encoding genes from A. niger and Penicillium crysogenum. A) Amino acid composition comparison. B) Homology tree representing the amino acid identities between the different amino acid sequences.

FIG. 10: Comparison of deduced amino acid sequences of partial glucosamine: fructose-6-phosphate aminotransferases encoding genes from A. niger, Penicillium crysogenum and Fusarium oxysporum A) Amino acid composition comparison. B) Homology tree representing the amino acid identities between the different amino acid sequences.

FIG. 11: Construction of a gfaA disruption mutant in Aspergillus niger. A) Schematic representation of the gfaA locus in A. niger. Bm=BamHI, Bg=BglII, X=XhoI, P=Pst1. The GFA coding region is indicated in black and the arrow indicates the direction of transcription. B) Southern blot analysis. Genomic DNA of a glucosamine requiring mutant (MA37.2.9) and a strain transformed with the pAO4-13 plasmid (AB4-13) was digested with PstI. The blot was hybridised with a 1.2 kb ClaI-BglII fragment containing part of the GFA gene. As expected, disruption of the GFA locus by the pyrG gene resulted in a loss of the 8.0 kb fragment which is present in a wild type strain and an appearance of a 7.0 kb, due to the presence of a PstI site in the pyrG gene of A. oryzae in the mutant strain. C) Morphological analysis of growth of the Δgfa strain in the presence or absence of glucosamine (glu). Fresh spores from a glucosamine requiring mutant (MA37.2.9) and control strain (AB4-13) were isolated from glucosamine containing agar plates and inoculated for 6 hours in the absence (C1 and C2) or in the presence (C3 and C4) of glucosamine (50 mg/ml).

### DETAILED DESCRIPTION OF THE INVENTION

The present invention is based on the elucidation of changes in the expression of genes in filamentous fungi after exposure to subtances that disturb cell wall biogenesis. In accordance with the present invention there are provided genes encoding enzymes related to the biosynthesis of the cell wall in filamentous fungi. Both an α-1,3-glucan synthase (agsA) from Aspergillus niger as well as its regulatory sequences, in particular its promoter sequence, and a Glutamine:fructose-6-phosphate aminotransferase (gfaA) from A. niger as well as its regulatory sequences, in particular its promoter sequence have been isolated. Both activities (α-1,3-glucan synthase activity and Glutamine:fructose-6-phosphate aminotransferase activity) are expected to be essential for fungal growth. We have shown that deletion of the gfaA gene in A. niger is lethal deleterious for growth. We have also shown that α-1,3-glucan synthase activity in A. niger is encoded by a family of at least four putative α-1,3-glucan synthase genes. Likely, single disruption of one of the ags genes agsA did not result in a growth defect because of the genetic redundancy. Because of the shown lethality of the gfaA deletion strain and the expected lethality of multiple ags deletion mutants, these two enzyme activities are well suited as a antifungal target for the development of new antifungal substances.

The Cell Wall as a Dynamic Structure

The fungal cell wall is a highly dynamic structure. Both its composition and architecture respond to external and internal stimuli. This response is best studied in S. cerevisiae where transcription of several enzymes involved in both chitin and β-glucan synthesis are highly regulated during the cell cycle (Shaw et al., 1991, Ram et al., 1995, Igual et al., 1996, Cabib et al., 1997, Smits et al., 1999). Also, the transcription of several cell wall protein encoding genes is cell cycle regulated (Caro et al., 1998). In addition to structural differences in cell wall composition during the cell cycle, evidence has emerged that also the formation of linkages between different cell wall components is cell cycle dependent (Hartland et al., 1994, Cabib et al., 1997).

Differences in cell wall architecture between different parts of the hyphae have also been observed in filamentous fungi. Cell wall growth of filamentous fungi occurs by atypical deposition of cell wall polysaccharides, especially chitin and 1,3-β-glucans. This has been shown by microscopic autoradiography with tritiated N-acetylglucosamine and glucose (Bartnicki-Garcia and Lippman, 1969, Gooday, 1971). In growing hyphae, 1,6-β-glucan linkages are formed behind the extreme apex, which is beyond the region of most active β-1,3-glucan synthesis (Sietsma et al., 1985, Sonnenberg et al., 1985). This crosslinking is important to give the cell wall its rigid structure. This is illustrated by the rapid bursting of apices of growing hyphae by antibiotics, such as nikkomycin and echinocandin, specific inhibitors of chitin and glucan synthesis, respectively (Gooday, 1990, Zhu and Gooday, 1992), or as the present inventors have found by treating mycelium with Novozyme® (a preparation of cell wall degrading enzymes containing α-and β-glucanases, chitinase and proteases).

Cell Wall Remodeling in Response to External Damage

Research in S. cerevisiae has shown that resistance to cell wall degrading enzymes or antifungal proteins is correlated with the expression of certain cell wall proteins. For example, it has been shown that a yeast mutant lacking certain cell wall proteins, such as Cwp2p or Sed1p, become more sensitive to Zymolyase, a mixture of β-1,3- and β-1,6-glucanases (Van der Vaart et al., 1995, Shimoi et al., 1998). Expression of specific cell wall proteins (Cwp1p and Cwp2p) also confer resistance to the antimicrobial peptide nisin (Dielbandhoesing et al., 1998). Furthermore, it has been shown that PIR proteins conveys resistance to tobacco osmotin, a antifungal protein of the PR-5 family. Yun et al., showed that expression of Pir2p is induced by osmotin and that overexpression of Pir proteins increases resistance towards osmotin, whereas deletion of all Pir genes resulted in sensitivity (Yun et al., 1997).

The presence of cell wall degrading enzymes or antifungals that interfere with cell wall biosynthesis will lead to cell wall damage and concomitant hypotonic stress. Hypertonic stress conditions can be mimicked in S. cerevisiae by using cell wall mutants. Cell wall mutants (e.g., fks1Δ and gas1Δ) with lower levels of β-1,3-glucan, are large and swollen, and thus probably under continuous osmotic stress. The hypotonic stress was shown to induce the expression of a cell wall protein (Cwp1p) and the synthesis of chitin and β-1,3-glucan (Ram et al., 1998). The different cell wall composition in these mutants is accompanied by massive changes in the molecular architecture of the cell wall (Kapteyn et al., 1997). Whereas in wild type cells only 2% of all CWPs are linked to chitin through β-1,6-glucan, this linkage becomes 20-fold more abundant in β-1,3-glucan deficient mutants. These cell wall damage activated changes seem to contribute to the strength of the cell wall as part of a general compensation mechanism to ensure cell wall integrity. Support for it comes from the observation that the increased deposition of chitin is important for cell survival in fks1Δ and gas1Δ mutants as the mutants show increased sensitivity to nikkomycin, an inhibitor of chitin synthase (El Sherbeiny et al., 1995; Popolo et al., 1997). Compensatory changes in cell wall composition have also been found in Aspergillus ssp treated with inhibitors of cell wall biosynthetic enzymes: drug-treated germlings showed a very thick cell wall with an extensive outer layer of cell wall mannoproteins at the hyphal tip (Kurtz et al., 1994).

Sensing Cell Wall Weakening

Several signal transduction pathways have been identified in S. cerevisiae that are likely to be involved in regulating cell wall remodeling when external conditions change (reviewed by Cid et al., 1995). It has been shown in S. cerevisiae that the PKC1-MPK1 signal transduction pathway regulates cell wall biosynthesis in response to low osmolarity conditions (Kamada et al., 1995). Putative upstream regulators have been identified recently and consist of a family of three integral membrane proteins (Wsc1-3p) that are localized to the plasma membrane (Verna et al., 1997). Disruption of the WSC genes result in phenotypes similar to the mutants in the PKC1-MPK1 pathway, namely, an osmotically remediable cell lysis phenotype (Verna et al., 1997 and references cited therein). Since Pkc1p is one of the targets for Rho1p (Nonaka et al., 1995, Kamada et al., 1996), and Rho1p is activated via the exchange factor Rom2p in response to cell wall stress (Bickle et al., 1998), the following mechanism emerges. Cell surface stress, either caused by mutations that affect cell wall biosynthesis, the presence of cell wall degrading enzymes, or low osmolarity conditions, results in swelling of the cells. Swelling corresponds with membrane stretch which might be sensed by mechanosensitive ion channels (Gustin et al., 1988) and/or the Wsc proteins. Wsc protein would then activate Rom2p which activates Rho1p. Rho1p can than activate the PCK1-MPK1 pathway which regulates gene expression of genes involved in cell wall biosynthesis such as Bgl2p, a transglucosylating enzyme (Shimizu et al., 1994), and Fks2p, a subunit of the β-1,3-glucan synthase complex (Zhao et al., 1998).

We have now found, after extensive research and experimentation, that similar cell wall remodeling mechanisms are present in certain filamentous fungi, in particular in (but not restricted to) Aspergillus species. We have identified a family of α-1,3-glucan synthase encoding genes and found that the expression of one of the members of this family is highly induced after cell wall stress by CFW treatment. This was unexpected and unpredictable since said enzyme is not present in the yeast S. cerevisiae. Similarly for what has been found in S. cerevisiae, we have also found that the mRNA for the enzyme glutamine:fructose-6-phosphate amino transferase is induced upon cell wall stress, indicating a role for chitin biosynthesis in the cell wall remodelling mechanism.

Based on these findings a method is provided of selecting and identifying sequences which are useful for the screening of potential new fungicides, comprising the following steps:
1) identification of genes involved in the architectural changes in the fungal cell wall such as genes encoding cell wall proteins and cell wall biosynthetic enzymes;
2) development of one or more suitable reporter constructs using promoter sequences of one or more induced genes to monitor the response of the cell to stress conditions;
3) identification of regulatory elements from said promoter sequence involved in regulation; and
4) evaluation of the use of the reporter constructs for the screening of antifungal compounds in other fungi.

In addition, a method is provided for the identification of new antifungal targets and the development of new reporter constructs for the identification of antifungal substrates in filamentous fungi, comprising the following steps:
1) identification of genes involved in the architectural changes in the fungal cell wall such as genes encoding cell wall proteins and cell wall biosynthetic enzymes;
2) use of the enzymatic activity of proteins encoded by said genes that are induced upon cell wall stress, which proteins contribute to cell wall biosynthesis and cell wall remodeling, as potential targets for new antifungals.
3) evaluation of the usefulness of identified genes encoding enzymatic activities involved in cell wall biosynthesis/remodeling as an antifungal target.

The present invention will now be described in some detail using Aspergillus niger as a typical example for a further description of the principles of the invention. It should be understood however, that the present invention is in no way restricted to aspergillus or genes or part of genes thereof.

Isolation of Partial Clones Encoding Cell Wall Biosynthesis Related Proteins from A. niger.

For the isolation of genes that are induced by the presence of sublethal concentrations of antifungals, various candidate genes were chosen which are expected to be involved in cell wall biosynthesis. These genes include partial DNA sequences of the gfaA homologue, encoding the enzyme glutamine:fructose-6-phosphate aminotransferase, involved in the biosynthesis of UDP-N-acetyl-glucosamine, fksA, encoding a putative subunit of the β-1,3-glucan synthase complex, agsA, agsB, agsC and agsD, four homologous genes encoding different putative α-1,3 glucan synthases.

Isolation of α-1,3-glucan Synthase Genes in Aspergillus niger.

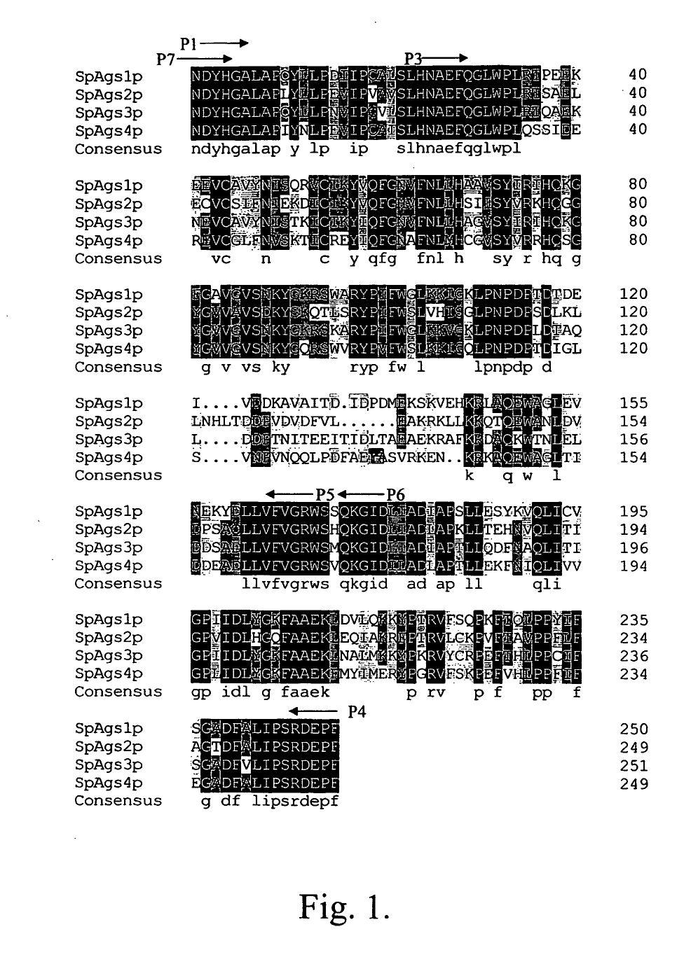

α-1,3-Glucan is an important cell wall component of several yeasts and fungi. However, this polymer is not present in the best studied yeast Saccharomyces cerevisiae. So far, only in the fission yeast Schizosaccharomyces pombe a family of genes encoding putative α-1,3-glucan synthases had been identified (Hochstenbach et al., 1998, Katayama et al., 1999). Sequence alignment of the several putative α-1,3-glucan synthases allowed the identification of conserved regions in which the amino acid sequence between the different synthases was identical. Based on these conserved regions, seven degenerated primers were designed, named AgsP1 to AgsP7, respectively, and used in Polymerase Chain Reactions (PCR) on A. niger genomic DNA to isolate putative α-1,3-glucan synthase encoding genes from A. niger (see Table 1 below, as well as FIG. 1).

TABLE 1

primers used for isolation of A. niger cell wall
biosynthetic enzymes

| | | |
|---|---|---|
| Primer | Protein | forward/ |

| | | | |
|---|---|---|---|
| name | DNA Sequence (5′-3′) | sequence | reverse |

| | | | |
|---|---|---|---|
| AgsP1 | AAYGAYTAYCAYGGNGC | NDYHGAL | forward |
| AgsP2 | WACCACCANCCNGGCAT | MPGWWF/Y | reverse |
| AgsP3 | CAYAAYGCNGARTTYCARGG | HNAEFQG | forward |
| AgsP4 | ARNCCRAANGGYTCRTC | DEPFLL | reverse |
| AgsP5 | CCANCKNCCNACRAANAC | VFVGRW | reverse |
| AgsP6 | ADRTCDATNCCYTTYTG | QKGIDL/I | reverse |
| AgsP7 | TAYCAYRTNAAYGAYTAYCA | YHV/INDYH | forward |
| GfaP1^{a} | CGGGATCCCGARTAYMGNGGNTAYGA | EYRGYD | forward |
| GfaP2^{a} | CGGAATTCCGTGNGTBGCCAANCKNGT | TRWATH | reverse |
| GfaP3^{a} | CGGGATCCCCAYATHAAYGCNGGNCC | HINAGP | forward |
| GfaP4^{a} | CGGAATTCCGCCYTGNARRCARTCNAC | VDCLQG | reverse |
| polyT | T20V | - | reverse |
| FksAP1 | GGNAAYCCNATHYTNGG | GNPILG | forward |
| FksAP2 | CCYTTNCCRCAYTGRWARTA | YY/FQCGKG | reverse |
| FksAP3 | GAYGCNAAYCARGAYAAYTA | DANQDNY | forward |
| FksAP4 | CCNGCRWADATRTCYTCRTT | NEDIY/FAG | reverse |

^{a}restriction site (EcoRI and BamH1) and additional nucleotides are shown in bold.

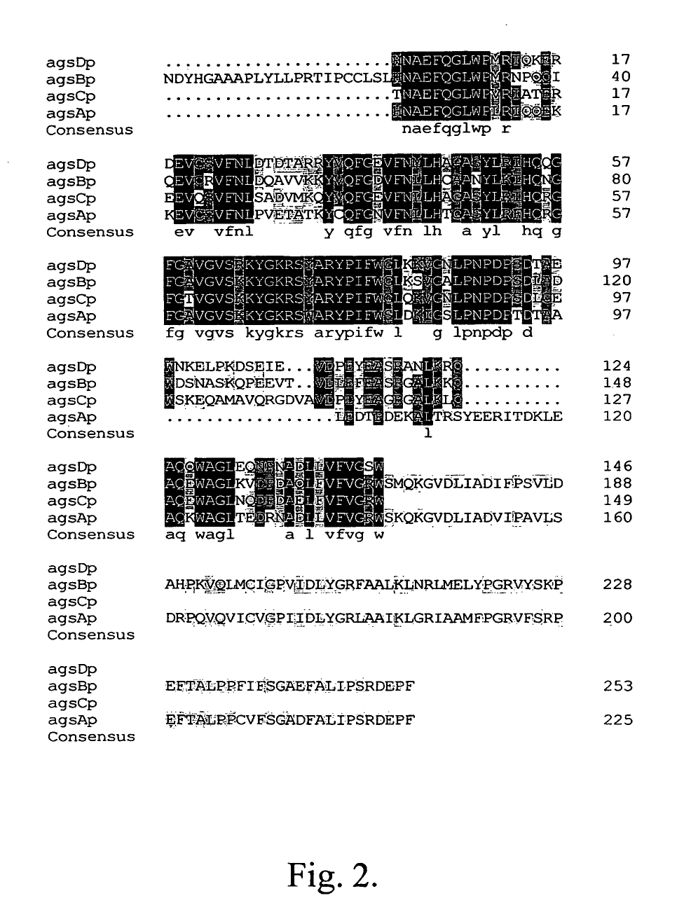

PCR fragments with the predicted length (448-822 bp) (depending on the primer combination and the presence of possible introns) were subcloned in pGEM-T^{easy} (Promega) and analysed by restiction enzyme digestion. After initial grouping, representatives of each group were sequenced. Sequence comparison revealed that 4 different putative α-1,3-glucan synthases encoding genes were isolated (FIG. 2) which were named agsA, agsB, agsC and agsD, respectively. Their sequences are represented in the Sequence Listing hereinafter and referred to as SEQ ID NOS: 1-2 (for agsA), SEQ ID NOS: 3-4 (for agsB), SEQ ID NOS: 5-6 (for agsC), and SEQ ID NOS: 7-8 (for AgsD), respectively.

Isolation of gfaA from A. niger

Sequence alignment of Gfa1p homologs from S. cerevisiae (Watzele and Tanner, 1989), S. pombe (Wood et al., 2002), C. albicans (Smith et al., 1996) and several higher eukaryotic GFA genes cloned (including GFA from human, mouse and from drosophila) revealed highly conserved stretches of amino acid sequences (Table 1). Degenerated primers were designed, based on the conserved amino acid sequences, and used to amplify the gfaA encoding gene fragment from the A. niger cDNA library. The first PCR reaction was carried out using primer GFAP4 and a polyT primer. A second ‘nested’ PCR was done on the products with primers GFAP4 and GFAP5. An expected PCR fragment of approximately 550 bp was cloned and sequenced. As expected, the amino acid sequence of the amplified fragment shows the higest identity towards the C. albicans protein (64% identity on amino acid level).

Isolation of fksA from A. niger

Sequence alignment of Fks1 homologues from S. cerevisiae (Douglas et al., 1994, Ram et al., 1995), S. pombe (Ishiguro et al., 1997), A. nidulans (Kelly et al., 1996) and A. fumigatus (Beauvais et al., 2001) revealed the presence of highly conserved amino acid stretches and four degenerated primers were obtained which allowed a nested approach for the isolation of the fksA homolog from A. niger using genomic DNA as a template for PCR. Amplified fragments with the expected size of 350 bp were cloned in PGEM-T^{easy} and sequenced. The deduced amino acid sequence was highly identital to the FKS proteins from Aspergillus species (94% identical) and yeast (84% identical). This indicates that the method disclosed in the present invention is broadly applicable.

Analysis of the Expression of Cell Wall Related Genes in Response to CFW

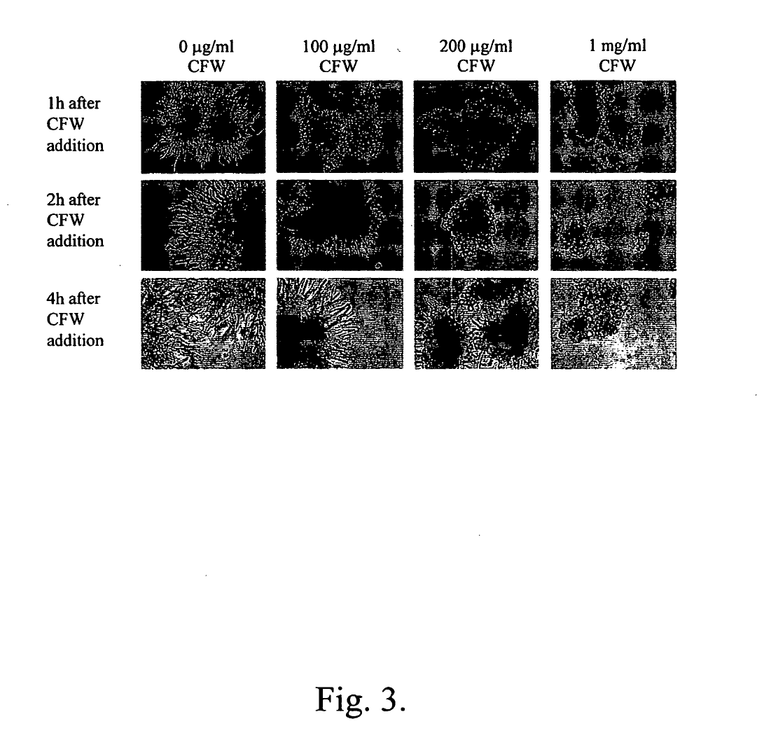

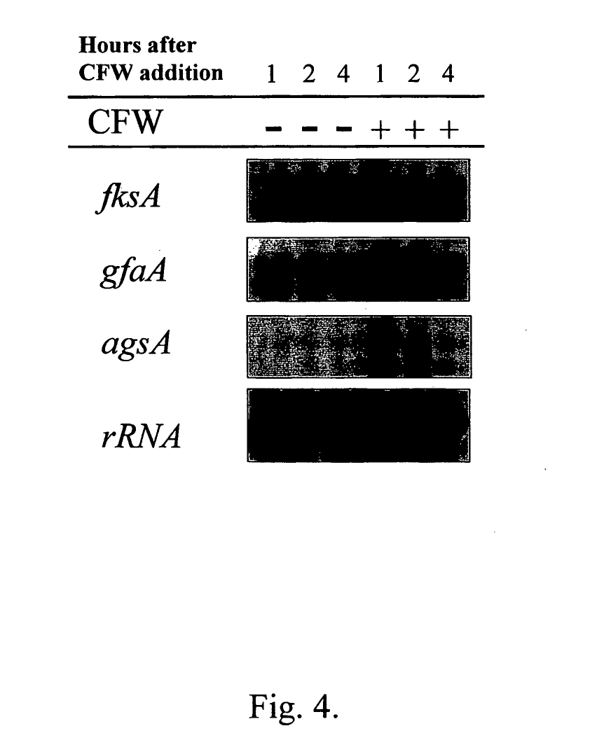

To identify genes that are induced upon cell wall stress, we have first investigated which cell wall perturbing compound was suited for this purpose. Finally, we used the antifungal compound Calcofluor White (CFW) to induce cell wall stress. Treatment of germinating spores with CFW resulted in the formation of swollen hyphe CFW binds to glycan fibers and thereby preventing their crystallization or crosslinking to other cell wall components. Because of the defects in cell wall assembly, the cell wall is weakened en swells from internal pressure. Polarized growth is resumed after 2-4 hours. Both the effect of hyphal tip swelling and the time that is required to resume growth are dependent on the concentration of CFW (FIG. 3). To identify genes that are induced upon CFW treatment, mRNA has been isolated from germling that have been treated with or without CFW for various time intervals. In a typical experiment as shown in FIG. 3. Fresh spores were grown for 5 hours at 37° C. which resulted in the formation of a small germ tube. These germlings were than treated with 200 μg/ml CFW. After 1, 2 and 4 hours after addition of CFW total RNA was isolated and used in Northern blot experiments (see FIG. 4). As a control, total RNA was isolated from germlings that were not treated with CFW. Several probes encoding putative proteins involved in cell wall biosynthesis have been analysed and quantified.

The expression level of fksA was not significantly altered in the presence of CFW. For two genes, gfaA and agsA, the expression was found to be increased after CFW addition. The highest induction was found for agsA. Thus, these genes are regulated by the promoter sequences as a response to the induced cell wall stress. Such promoters are suitable candidates for use as reporters. Furthermore, the genes induced upon cell wall stress are expected to encode proteins that contribute significantly to cell wall biosynthesis and cell wall remodeling. As stated before, the enzymatic activities of those proteins are potential targets for new antifungals. Quantifying the signal showed that the increase in mRNA level was at least 20-fold for agsA and 4-fold for gfaA. No signal could be detected for the other ags homologs.

Cloning of Both the Full Length agsA Gene and the gfaA Gene from A. niger

From an existing cosmid library (TNO Nutrition, Zeist, the Netherlands), approximately 5000 cosmids clones were spotted on nylon filters. Clones were grown over night and lysed by standard procedures (Maniatis et al., 1987). From this ordered genomic cosmid library, cosmids clones containing the agsA and the gfaA genes were identified using the agsA and gfaA PCR amplified fragments as a probe.

Two cosmids that hybridized with the agsA probe were isolated and one of them (RD1.4) was characterized by restriction analysis. The cosmid was digested with various restriction enzymes and fragments were analysed by Southern blotting with the agsA probe. Several fragments These subclones were used for the sequencing of the agsA gene with sequences using vector primers to sequence the border sequences of the subclones. Based on these sequences oligonucleotides were designed to sequence the complete genomic DNA region containing the agsA gene. The complete DNA sequence encoding the agaA protein and 2.7 kb of promoter sequences was determined and is shown as SEQ ID NOS:9-13 in the sequence listing at the end of this specification.

The complete gfaA encoding gene was isolated in a similar way based on the amino acid similarity with gfa homologs from other organisms mentioned above. A cosmid clone, gfaA#5 was isolated and analysed by subcloning and sequence analysis. The complete gfaA encoding region and 1072 bp of the promoter sequence were then determined and is shown below as SEQ ID NOS:14-19.

Development of Various Reporter Constructs Useful for the Identification of Antifugal Compounds

The promoter sequence of the agsA gene has been used to set up several reporter systems by cloning genes encoding reporter proteins (e.g., acetamidase, Green Fluorescent Protein or GUS (beta-glucoronidase) behind the agsA promoter. An A. niger strain containing the reporter fusion constructs can be used in high-throughput screens to identify cell wall perturbing compounds. This approach allows the rapid screening of a huge number a potential anti-cell wall agents using different high throughput screens.

Construction of the PagsA-AmdS Reporter Strain.

Aspergillus niger grows poorly on acetamide as a nitrogen source. When additional copies of the amdS gene, encoding a acetamidase of A. nidulans are introduced, A. niger can grown on acetamide plates and the amdS gene can therefore be used as a selectable marker (Kelly and Hynes, 1985). The coding region of the amdS gene was fused to the agsA promoter sequence. Both the relatively low basal expression of the agsA gene and the high level of induction after CFW stress (over 20-fold) makes the agsA promoter a good candidate for use as a reporter. The reporter construct further comprises the amdS gene (A. nidulans) with its own terminator. For targeting to the pyrG locus of the A. niger strain AB4.1 (pyrG⁻) the pyrG* gene is included in the construct (cf. Van Gorcum et al., 1988).

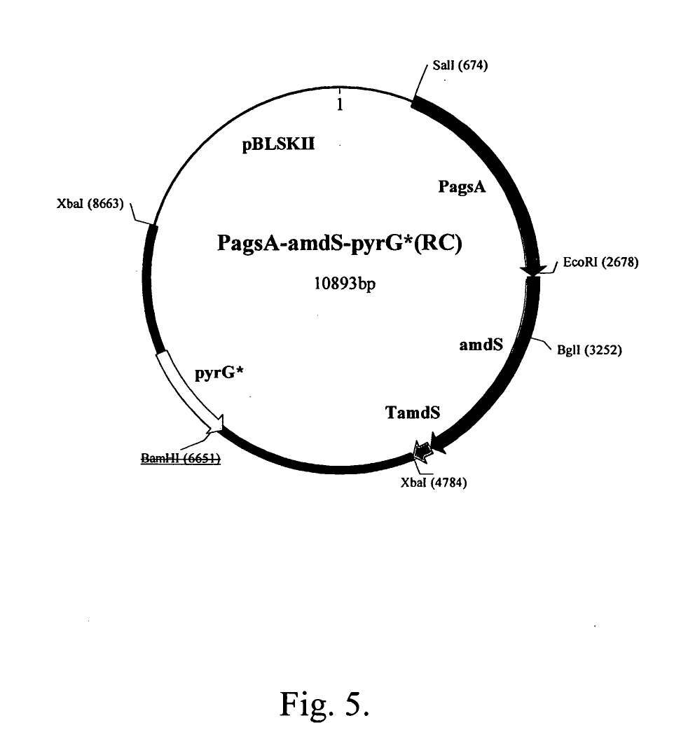

Construction: A 2010 bp SalI-EcoRI fragment containing the promoter region of the agsA gene (FIG. 5) was isolated from pRD12 and ligated into a SalI-EcoRI digested pBluescript SKII vector (Stratagene). This vector was re-digested with EcoRI-XbaI and used in a three-way ligation. The second fragment of the three-way ligation is a 587 bp EcoRI-BglI fusion of 31 bp (introduced by the primer) of the agsA promoter and the first part of the amdS gene. This fragment was amplified by PCR using two primers (AmdS-agsAP1 5′-CACAGAATTCCTGGTACCACACGCCGCTTGCCATCATGCCTCMTCCTGGGAAG (EcoRI site underlined and ATG startcodon in bold) and AmdS-agsAP2 5′-GCCATGAGATGTAGCCCATTG) using plasmid P3SR2 (Corrick et al., 1987) as a template. The fragment was cloned into pGEM-T-easy (PROMEGA) and verified for PCR introduced mutations by DNA sequence analysis. The last fragment of the three-way ligation was a 1544 bp BglI-XbaI fragment containing the last part of the amdS gene and the amdS terminator from p3SR2 (Corrick et al., 1987). After ligation the vector was opened with XbaI and a 3879 bp XbaI fragment containing the pyrG* gene (Van Gorcom et al., 1988) was ligated into the vector to give PagsA-amdS-PyrG* (FIG. 5).

Transformation and Analysis of the Transformants

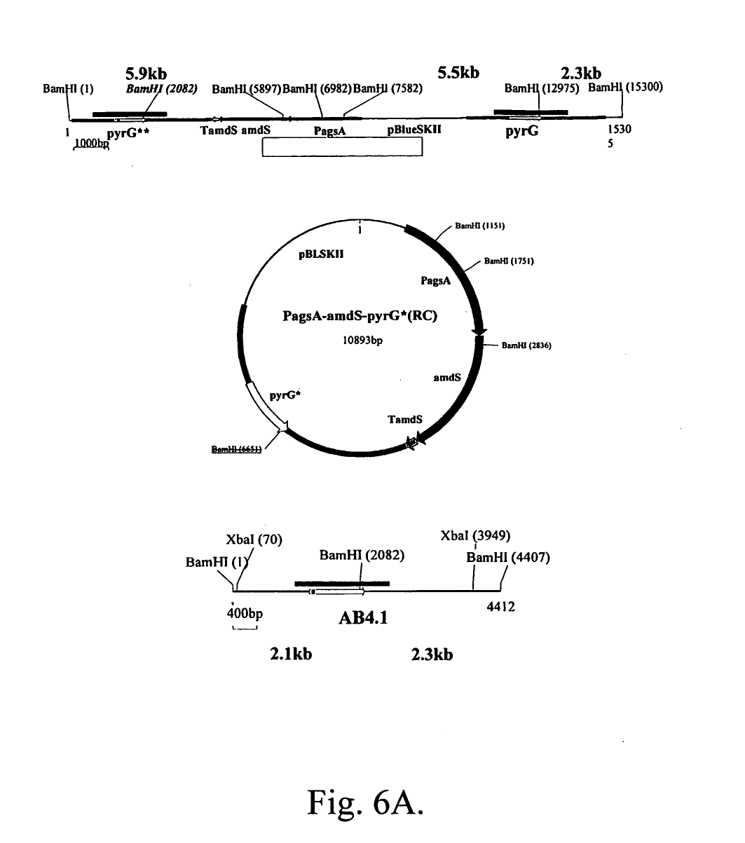

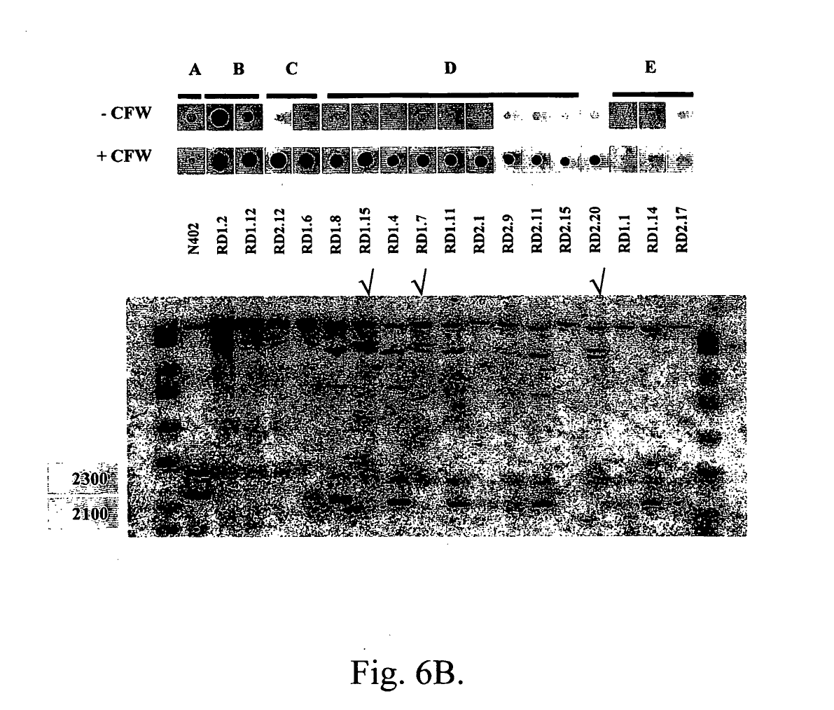

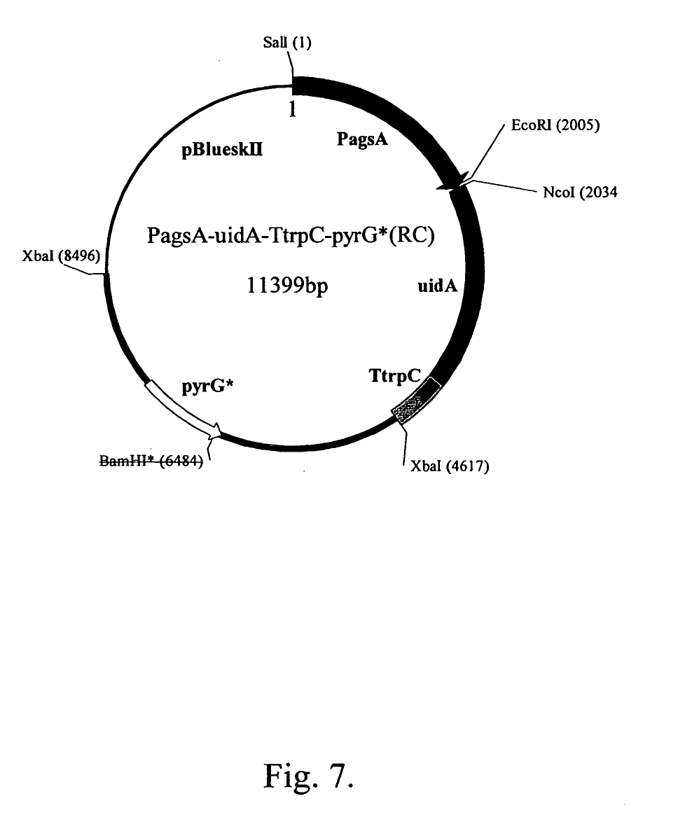

The construct was transformed to strain AB4.1 (a pyrG negative strain derived from N402) and transformants were selected on the presence of an intact pyrG gene. Wild type strain N402 and purified transformants (ABRD1.1-1.25 and ABRD2.1-2.15) were analysed were grown on acetamide and acrylamide plates. As expected, the wild type strain and most of the transformants did not grow on acetamide plates as expected from the low basal activity of the agsA promoter (FIG. 6). Only two transformants RD1.2 and RD1.12 could grow on these aceetamide. Southern blot analysis of these transformants (FIG. 7b) showed that these strains obtained multiple copies of the plasmid which explains that the strains could grow on acetamide plates. This result demonstrate that the expression of the agsA gene under control of its own promoter under normal growth conditions is too low for an efficient growth on acetamide plates. Next, it was examined if the addition of CFW in the medium could activate the agsA promoter and thereby allow growth on acetamide plates. As can be seen in FIG. 7b the addition of 0.5 mg/ml CFW to the plates resulted in growth (and sporulation) for the majority of the transformants. The majority of the transformants were analysed by Southern blotting to confirm correct integration of the construct. Among others, strain RD1.7 showed the correct single copy integration pattern.

The results show that this strain is suitable for screening for antifungal compounds that are related to disturbing cell wall biosynthesis and assembly. We have shown that a sublethal concentration of CFW activates the agsA promoter and thereby induce the expression of the amdS gene, allowing the strain to grow on acetamide.

Construction of the PagsA-GUS Reporter Strain

To quantify the expression of the agsA promoter in response to the presence of cell wall disturbing compounds, a reporter construct containing the (β-glucuronidase (GUS) gene (uidA) behind the agsA promoter. For termination the trpC terminator was used. Since GUS activity is easily detectable using X-glc as a substrate and A. niger lack endogenous GUS-activity this reporter protein was used.

Construction: The three-way ligation as described for the PagsA-amdS construct was repeated. The only difference was that the PCR reaction to amplify the fragment that consisted of the 31 bp of the agsA promoter and the first 587 bp of the amdS gene was performed with a different primer that also introduced a NcoI site on the ATG (AmdS-agsAP3-NcoI 5′CACAGAATTCCTGGTACCACACGCCGCTTGCCACCATGGTCCCTCAA TCCTGGGAAG (EcoRI and NcoI sites are underlined and the ATG is in bold).

After ligation, to give PagsA-amdS-ncoI, a 4943 bp XbaI-NcoI fragment containing the promoter of agsA and the Pbluescript SK vector was isolated and used in a three-way ligation with two other fragments. One of the other fragments was isolated as a 1866 bp NcoI-SalI fragment containing the complete uidA gene from pNOM102 (Z32701). The third fragment of the three-way ligation was a 729 XhoI-XbaI fragment containing the trpC terminator. This fragment was derived after cloning a 719 bp BamHI-XbaI fragment from pAN52-1not (Z32697) in puc21 (AF223641). The fragment was re-isolated from this plasmid after XhoI-XbaI digestion. After ligation the vector (PagsA-uidA-TtrpC) was re-opened with XbaI and a 3879 bp XbaI fragment containing the pyrG* gene was ligated into the vector to give PagsA-uidA-TtrpC-pyrG* (see FIG. 7a).

Transformation and Analysis of the Transformants

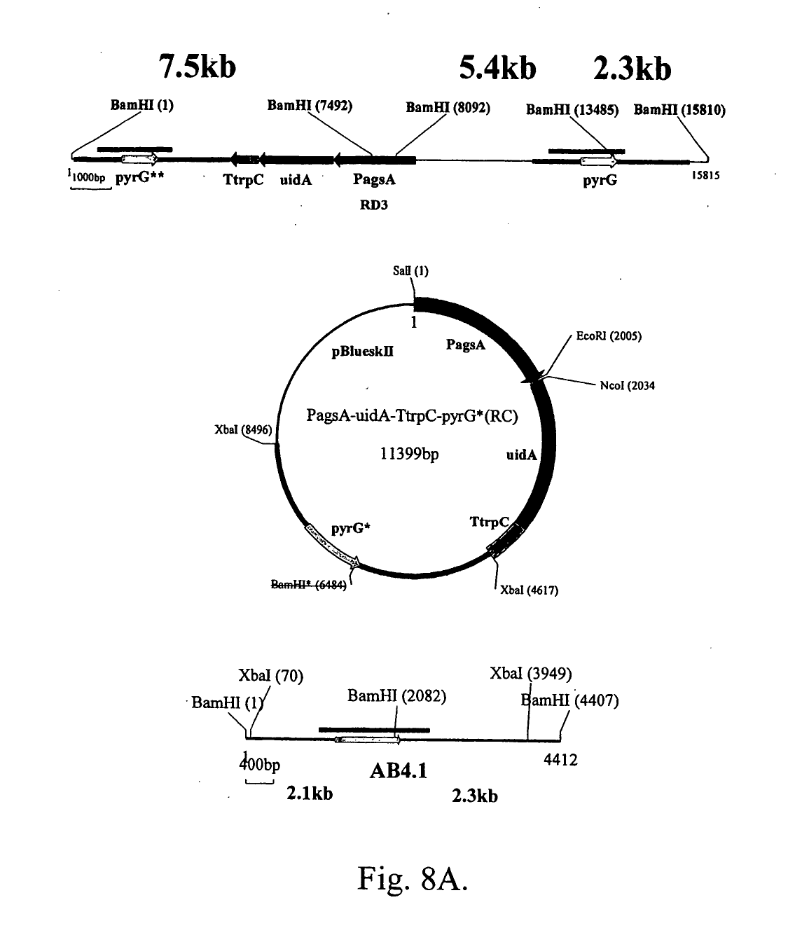

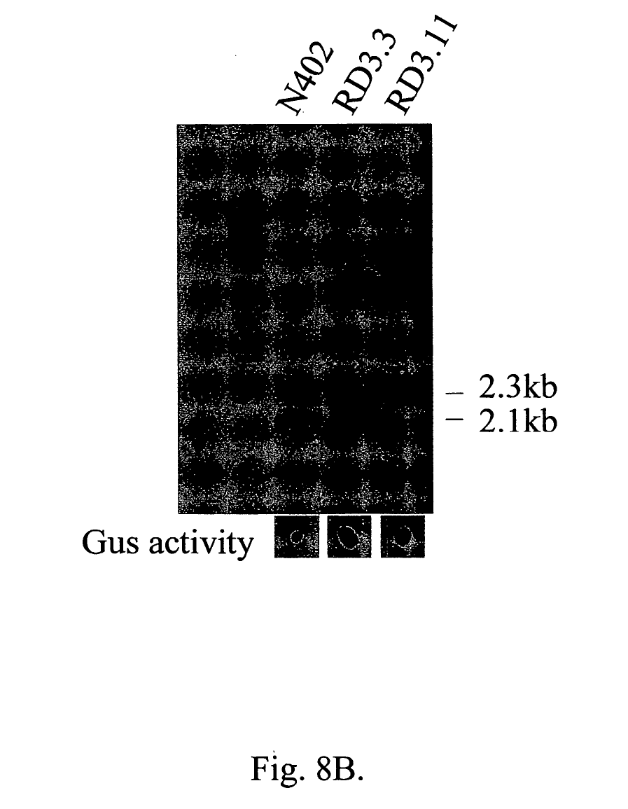

The disruption vector was transformed to A. niger strain AB4.1 and pyrG* transformants have been purified and analysed on Southern blot to select single copy integrants. FIG. 8b shows two transformants with the predicted DNA pattern after single copy integration. These strains can be used to measure to screen for substances that induce GUS expression.

Isolation of ags Homologs Genes from Other Fungi

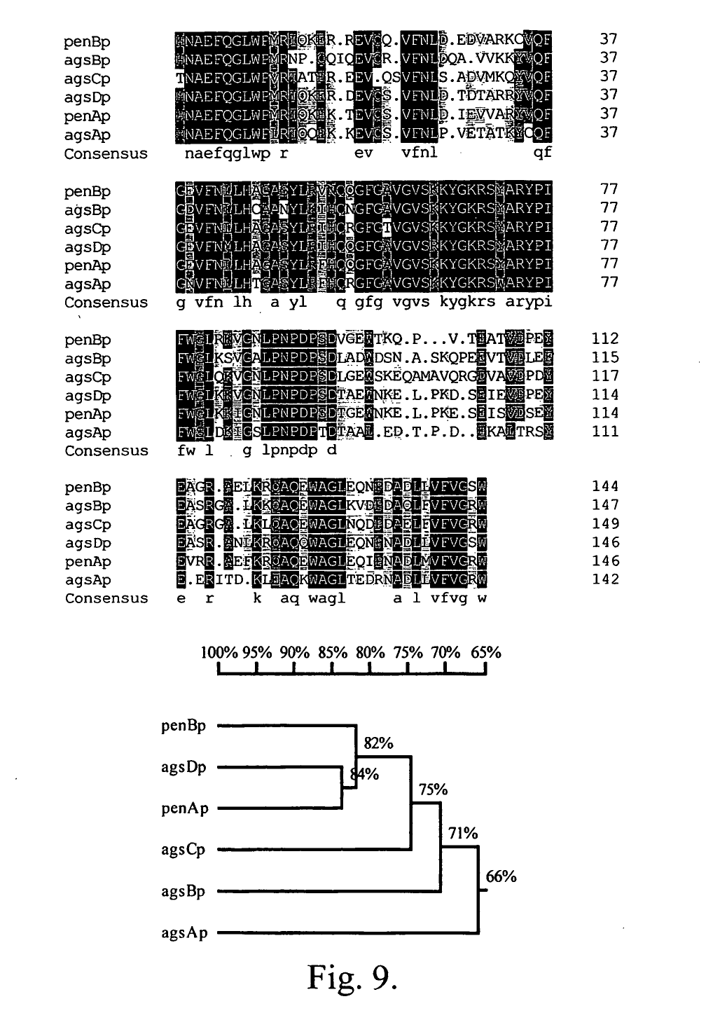

The degenerated primers that were used to isolate ags genes from A. niger, were also used to isolate ags genes from the food spoilage fungus Penicillium crysogenum. PCR fragments of the expected size were isolated and sequenced. Sequence analysis revealed that three ags genes from Penicillium were isolated, named PCAgsA, PCAgsB and PCAgsC, which are shown as SEQ ID NOS:20-21 (PCAgsA), SEQ ID NOS:22-23 (PCAgsB), and SEQ ID NOS:24-25 (PCAgsC), respectively. The amino acid sequence alignment of the different ags genes from A. niger and Penicillium is shown in FIG. 9.

The isolation of several ags genes from different filamentous fungi has been shown and it is our fair expectation that alpha-1,3-glucan genes are present in other filamentous fungi as well and that the induction of one of the ags genes is a general mechanism to deal with cell wall stress conditions.

Therefore, the present invention is in no way restricted to the Aspergillus niger example detailed above, nor to Aspergillus species. The principle disclosed herein also pertains to other filamentous fungi, including plant pathogenic fungi, e.g. Fusarium oxysporum ssp, Fusarium solani, Cladosporium fulvum, and Magnaporthe grisea, food spoilage fungi, e.g Penicillium ssp., and the medically important fungus Aspergillus fumigatus.

Isolation of gfa Homologs Genes from Other Fungi

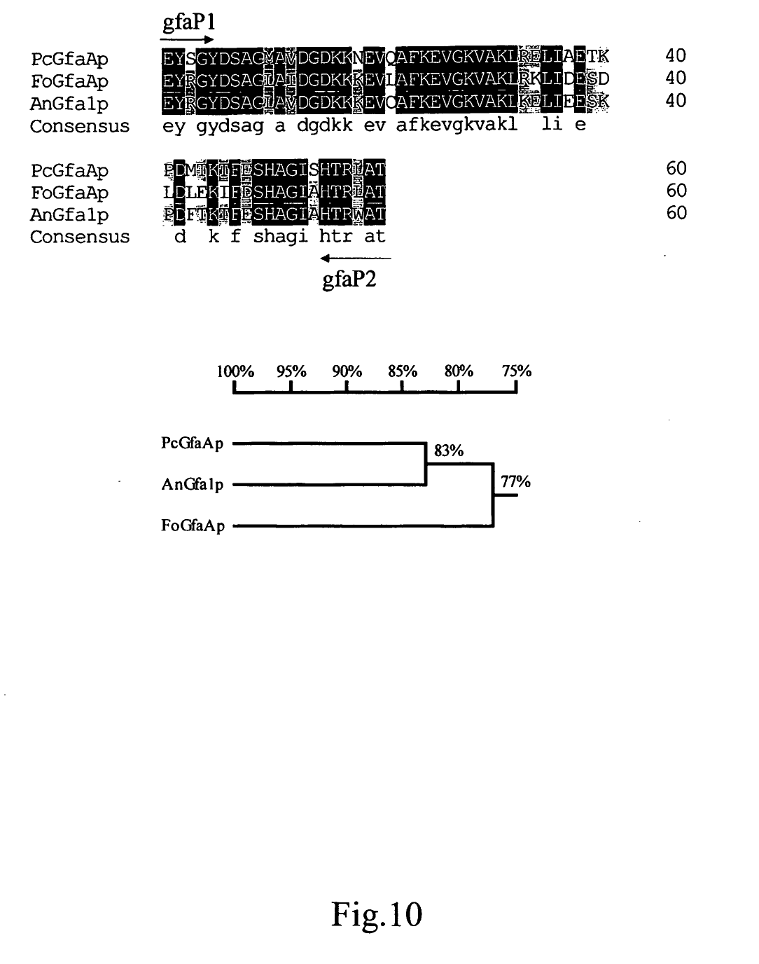

Sequence alignment of Gfa1p homologs from S. cerevisiae (Watzele and Tanner, 1989), S. pombe (Wood et al, 2002), C. albicans (Smith et al., 1996) and several higher eukaryotic GFA genes cloned (including GFA from human, mouse and from drosophila) revealed highly conserved stretches of amino acid sequences. Degenerated primers GFAP1 and GFAP2 (Table 1) were designed and used to amplify the gfaA encoding gene fragment from genes from the plant plantogenic fungus Fusaridum oxysporum f. sp. radicis lycopersici and the food spoilage fungus Penicillium crysogenum. Expected ˜200 bp PCR fragments obtained after PCR using genomic DNA from Fusarium and Penicillium as template DNA, respectively, were cloned and sequenced in the same way as described above. The Fusarium and Penicillium genes were named FUGfaA and PCGfaA, respectively, and their sequences are shown below in SEQ ID NOS:26-27 and SEQ ID NOS:28-29, respecitvely. The predicted amino acid sequences of the DNA sequences showed, as expected, a high degree of identity between the fungal species (FIG. 10). Thus, the successful isolation of a part of the gfaA encoding genes from filamentous fungi was demonstrated. Therefore, the methods disclosed in the present invention are broadly applicable to filamentous fungi in general.

GfaA is Essential for Viability in Aspergillus niger.

We have shown that both a gene encoding an α-1,3glucan synthase (agsA) and a gene encoding a glutamine:fructose-6-phosphate (gfaA) are induced after cell wall stress. The induction suggests an important function of the proteins in the process of cell wall biogenesis particularly under stress conditions. Therefore, both enzyme activities are expected to be required for proper cell wall biosynthesis and/or cell wall remodeling in response to conditions that are harmful for the cell wall. Because of their expected essential function, these enzyme activities are good candidates as targets for antifungal substances. In order to investigate the consequences of a loss of function of the gfaA activity in A. niger vector pDgfaA was constructed to disrupt the gfaA gene in A. niger by replacing said gene with the pyrG gene from A. oryzae.

Construction of the Disruption Plasmid

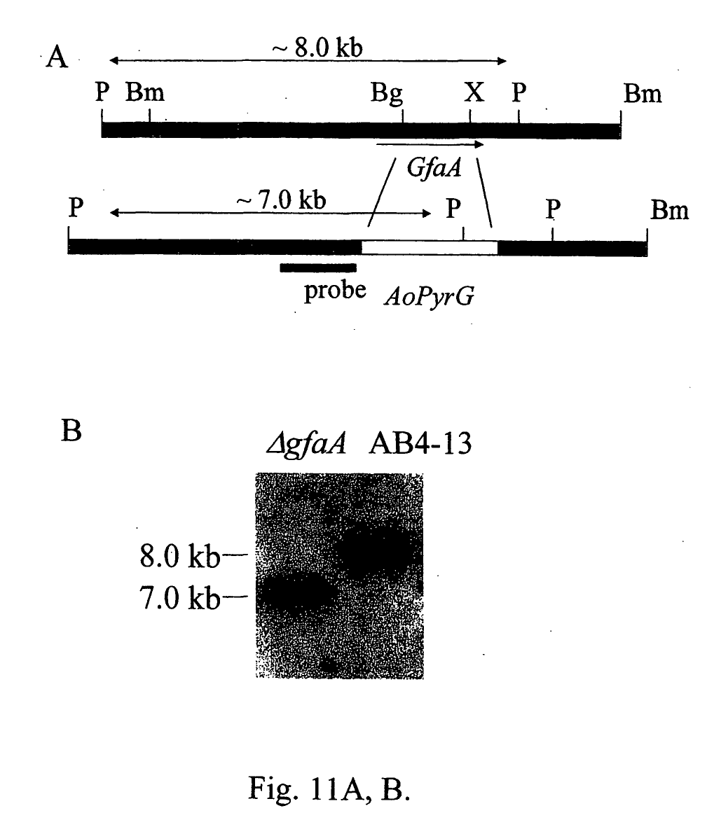

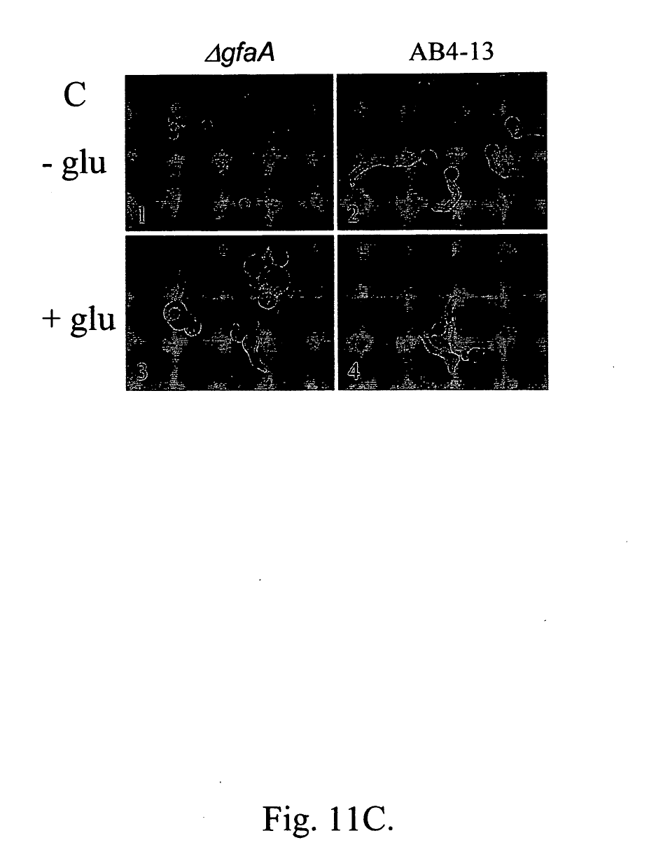

From cosmid clone GFA#5, a 9 kb BamHI subclone was made in vector pBluescript SK (Stratagene). From this vector, a ˜5 kb NotI-BglII fragment was isolated containing the 5′ promoter sequence of the gfaA gene. (the NotI site is from the Pbluescript cloning vector) A 3.0 kb BamHI-SalI fragment containing the A. oryzae pyrG gene was obtained from pAO4-13 (unpublished vector from own collection). These two fragments were used in a three way ligation using the 9 kb BamHI subclone which was digested with NotI and XhoI thus containing the 3′ terminator sequence and the vector (FIG. 11A). Proper ligation of the fragments was verified by restriction enzyme digestions and the plasmid, pDgfaA was used in a gene disruption experiment.

Disruption of gfaA

An A. niger pyrG, negative strain (AB4.1) was transformed with the BamHI linearised 12 kb fragment of the disruption casette. Transformants were selected on minimal medium supplemented with 5 mg/ml glucosamine. 40 transformants were obtained and subjected to two rounds of purification. Next, the transformants were grown on plates lacking glucosamine and true ΔgfaA transformants were expected not to grow on these plates. Indeed, such transformants were found. These glucosamine requiring mutants were further analysed by Southern blot analysis which proved that the expected deletion of the gfaA gene had occurred FIG. 11B. To determine the effect of gfaA deletion on spore germination, spores of a ΔgfaA strain were isolated after growth on a plate supplemented with glucosamine. Subsequently, these spores were inoculated in glucosamine free medium which resulted in a severe defect in spore germination (FIG. 11C-1). Some spores started to swell, but failed to form a germination tube which was normally formed in the wild type strain (FIG. 11C-2). Supplementation of the ΔgfaA strain with glucosamine (5 or 50 mg/ml) did not result in a complete restoration of the growth behaviour like the wild type strain, but allowed the formation and the outgrowth of a germ tube (cf. FIGS. 11C-3 and 4).

In conclusion, it was demonstrated that deletion of the gfaA gene is essential for growth of A. niger. The fact that glucosamine-6-phosphate tranferase activity is required for growth in A. niger makes it an attractive target for antifungal compounds.

Methods to Set Up HTS to Measure the Effect of Substances on the Glucosamine-6-phosphate Tranferase Activity and the α1,3 Glucan Synthase Activity.

The obtained nucleotide sequences encoding filamentous fungal proteins having α-1,3-glucan synthase activity and glucosamine-6-phosphate tranferase activity, respectively, can be used to search for new antifungal substances that interfere with or inhibit the enzymatic activities of said enzymes. Overexpression and purification of the said enzymes in appropiate hosts (E. coli, P. pastoris) and purification of the enzymes allows the development of an in vitro High throughput screening assay to measure said enzyme activities. Development of such assays allows the screening and identification of substances that interfere with or inhibit the enzymatic activities of said enzymes.

### REFERENCES

Bartnicki-Garcia, S. and E. Lippman. (1969). Science 165: 302-304.
Beauvais, A., Bruneau J. M., Mol P. C., Buitrago M. J., Legrand, R and J-P Latge. (2001). J. Bacteriology 183: 2273-2279.
Bickle, M., P. A. Delley, A. Schmidt and M. N. Hall. (1998). EMBO J. 17: 2235-2245.
Borgia P. T. and C. L. Dodge. (1992). J. Bacteriol. 174: 377-83.
Brul, S., King, A., Van der Vaart, M. J., Chapman, Klis, F. M., and C. T. Verrips. (1997). Antonie van Leeuwenhoek 72: 229-237.
Cabib, E. , T. Drgon, J. Drgonova, R. A. Ford and R. Kollar. (1997). Biochem. Soc. Transact. 25: 200-204.
Cao, C., Chan, C., Lee, C., Wong, S. S., and K. Yuen. (1998). Infect. Immun. 66: 966-973.
Caro, L. H. P., G. Smits, P. van Egmond, H. van den Ende and F. M. Klis. (1998). FEMS Microbiol. Lett. 161: 345-349.
Corrick, C. M., Tworney, A. P., and M. J. Hynes. (1987). Gene 53: 63-71.
Dielbandhoesing, S. K., H. Zhang, L. H. P. Caro, J. M. van der Vaart, F. M. Klis, C. T. Verrips, and S. Brul. (1998). Appl. Environ. Microbiol. 64: 4047-405.
Douglas et al. (1994). PNAS 91: 12907-12911.
El-Sherbeini, M. and J. A. Clemas. (1995). Antimicrob. Agents Chemother. 39: 200-207.
Gooday, G. M. (1971). J. Gen. Microbiol. 67:125-133.
Gooday, G. M. (1990). Inhibition of chitin metabolism. pp 61-79. Springer-Verlag: Berlin.
Gooday, G. and D. Schofield. (1995). Can. J. Bot. 73:114-121.
Gooday, G. W. (1995). Cell walls. pp 43-62. Chapman & Hall, London.
Hartlant, R. P., C. A. Vermeulen, F. M. Klis, J. H. Sietsma and J. G. H. Wessels. (1994). Yeast 10:1591-1599.
Igual, J. C., A. L. Johnson and L. H. Johnston. (1996). EMBO J. 16: 5001-5013.
Ishiguro, J. Saitou, A., Duran, A. and Ribas, J. C. (1997). J. Bacteriology 179: 7652-7662.
Kamada. Y., U. S. Jung, J. Piotrowski and D. E. Levin. (1995). Genes Dev. 9:1559-1571.
Kamada, Y., H. Qadota, C. P. Python, Y. Anraku, Y. Ohya and D. E. Levin. (1996). J. Biol. Chem. 271: 9193-9196.
Kapteyn, J. C., R. C. Montijn, G. J. P. Dijkgraaf and F. M. Klis. (1994). Eur. J. Cell. Biol. 65: 402-407.
Kapteyn J. C., R. C. Montijn, G. J. P. Dijkgraaf, H. van den Ende and F. M. Klis. (1995). J. Bacteriol. 177: 3788-3792.
Kapteyn, J. C., A. F. J. Ram, E. M. Groos, R. Kollar, R. C. Montijn, H. Van Den Ende, A.
Llobell, E. Cabib and F. M. Klis. (1997). J. Bacteriol. 179: 6279-6284.
Kelly, R., Register, E., Hsu, M.-J., Kurtz, M., and Nielsen J. (1996). J. Bacteriol. 178: 4381-4391
Kelly J. M. and M. J. Hynes. (1985). EMBO J. 4: 475-479.
Kollar, R., B. B. Reinhold, E. Petrkova, H. J. C. Yeh, G. Ashwell, J. Drgonova, J. C. Kapteyn, F. M. Klis and E Cabib. (1997). J. Biol. Chem. 272:17762-17775.
Kurtz, M. B., I. B. Heath, S. Dreikorn, J. Onishi and C. Douglas. (1995). Antimicrob. Agents Chemother. 38: 1480-1489.
Montijn, R. C., J. van Rinsum, F. A. van Schagen and F. M. Klis. (1994). J. Biol. Chem. 269:19338-19342.
Montijn R. C., P. Van Wolven, S. De Hoog and F. M. Klis. (1997). Microbiol. 143:1673-1680.
Mrsa, V., T., Seidl, M., Gentzsch and W. Tanner. (1997). Yeast 13: 1145-1154.
Nonaka, H., K. Tanaka, H. Hirano, T. Fujiwara, H. Kohno, M. Umikawa, A., Mino and Y. Takai. (1995). EMBO J. 14: 5931-5938.
Popolo, L., D., Gilardelli, P., Bonfante and M. Vai. (1997). J. Bacteriol. 179: 463-469.
Ram A. F. J., S. S. C. Brekelmans, L. J. W. M. Oehlen and F. M. Klis. (1995). FEBS Lett. 358: 165-170.
Ram, A. F. J., J. C. Kapteyn, R. C. Montijn, L. H. P. Caro, J. E. Douwes, W. Baginsky, P. Mazur, H. van den Ende and F. M. Klis. (1998). J. Bacteriol. 180: 1418-1424.
Ram, A. F. J., H. van den Ende, and F. M. Klis. (1998). FEMS Microbiol. Lett. 162: 249-255.
Schoffelmeer, E. A. M., J. C. Kapteyn, R. C. Montijn, B. C. Cornelissen and F. M. Klis. (1996). Glucosylation of fungal cell wall proteins as a potential target for novel antifungal agents. In: Modern Fungicides and Antifungal Compounds
Sela-Buurlage, M. (1996). Thesis. Agricultural University of Wageningen.
Sentandreu, R., S., Mormeneo, and J. Ruiz-Herrera. (1994). Wessels and Meinhart (Eds.) 111-124.
Shaw, J. A., P. C. Mol, B. Bowers, S. J. Silverman, M. H. Valdivieso, A. Duran and E. Cabib. (1991). J. Cell. Biol. 114: 111-23.
Shimizu, J, K. Yoda and M. Yamasaki. (1994). Mol. Gen. Genet. 242: 641-648.
Shimoi, H., H., Kitagaki, H., Ohmori, Y., Iimura and K. Ito. (1998). J. Bacteriol. 180: 3381-3387.
Sietsma, J. H., A. S. M. Sonnenberg and J. G. H. Wessels. (1985). J. Gen. Microbiol. 161: 1331-1337.
Sonnenberg, A. S. M., J. H., Sietsma and J. G. H. Wessels. (1985). Exp. Mycol. 9: 141-148.
Stintzi, A., T. Heitz, V. Prasad, S. Wiedemann-Merdinoglu, S. Kauffmann, P. Geoffroy, M. Legrand and B. Fritig. (1993). Plant ‘pathogenesis-related’ proteins and their role in defense against pathogens. Biochimie 75: 687-706
Toh-e, A., S., Yasunaga, H., Nisogi, K., Tanaka, T., Oguchi and Y. Matsui. 1993. Yeast 9: 481-494.
Van Der Vaart J. M., L. H. P. Caro, J. W. Chapman, F. M. Klis and C. T. Verrips. (1995). J. Bacteriol. 177: 3104-3110.
Van Gorkum and C. A. M. J. J. van den Hondel. (1988). Nucleic Acids Res. 26: 9052
Verna, J., A. Lodder, K. Lee, A. Vagts and R. Ballester. (1997). Proc. Natl. Acad. Sci. USA. 94: 13804-13809.
Wood et al. (2002). Nature 415: 871-880.
Yun, D., Y. Zhoa, J. M. Pardo, M. L. Narasimhan, B. Damsz, H. Lee, L. R. Abab, M. Paino D'Urzo, P. M. Hasegawa and R. A. Bressan. (1997). Stress proteins on the yeast cell surface determine resistance to osmotin, a plant antifungal protein. Proc. Natl. Acad. Sci. USA 94: 7082-7087.
Zhao, C., U. S. Jung, P. Garrett-Engele, T. Roe, M. S. Cyert and D. E. Levin. (1998). Mol. Cell. Biol. 18:1013-1022.
Zhu, W. Y., and G. M. Gooday. (1992). Mycol. Res. 96: 371-377

1-29. (canceled)

30. An isolated nucleotide sequence, comprising a promoter sequence isolated from a genome of a filamentous fungus, wherein said promoter is capable of being activated by an agent that disturbs cell wall synthesis.

31. The isolated nucleotide sequence according to claim 30, wherein said filamentous fungus is a filamentous fungus selected from the species Aspergillus, Penicillium, Cladosporium, Magnaporthe, or Fusarium.

32. The isolated nucleotide sequence according to claim 31, wherein the filamentous fungus is Aspergillus niger.

33. The isolated nucleotide sequence according to claim 30, wherein said promoter sequence comprises a promoter sequence of a glutamine-fructose-6-phosphate amidotransferase gene.

34. The isolated nucleotide sequence according to claim 33, further comprising a coding region of a glutamine-fructose-6-phosphate amidotransferase gene of a filamentous fungus.

35. The isolated nucleotide sequence according to claim 30, wherein said promoter sequence comprises a promoter sequence of an α-1,3-glucan synthase gene.

36. The isolated nucleotide sequence according to claim 35, further comprising a coding region of an α-1,3-glucan synthase gene of a filamentous fungus.

37. A nucleotide sequence of claim 30, further comprising a reporter gene operably linked to the promoter.

38. The reporter construct of claim 37, wherein the promoter sequence is a promoter sequence of an α-1,3-glucan synthase gene or a glutamine-fructose-6-phosphate amidotransferase gene.

39. The reporter construct of claim 37, further comprising a selectable marker.

40. The reporter system of claim 37, wherein the reporter gene is a coding region for an acetamidase, Green Fluorescent Protein, or a beta-glucoronidase.

41. A method for identifying an agent that disrupts cell wall synthesis in a filamentous fungus, comprising:
(a) introducing a reporter construct into a cell, wherein said reporter construct comprises a promoter of a filamentous fungus operably linked to a reporter gene, and wherein said promoter is capable of being activated by an agent that disrupts cell wall synthesis in the filamentous fungus;
(b) exposing the cell to the agent; and
(c) determining whether said reporter gene is expressed.

42. The method according to claim 41, wherein said reporter construct further comprises a selectable marker.

43. The method according to claim 41, wherein said promoter comprises an α-1,3-glucan synthase promoter or a glutamine-fructose-6-phosphate amidotransferase promoter.

44. The method according to claim 41, wherein said reporter gene comprises a coding region for an acetamidase, Green Fluorescent Protein, or a beta-glucoronidase.

45. The method according to claim 41, wherein said filamentous fungus is a filamentous fungus selected from the species Aspergillus, Penicillium, Cladosporium, Magnaporthe, or Fusarium.

46. The method according to claim 41, wherein said filamentous fungus is Aspergillus niger.

47. An isolated nucleotide sequence comprising a promoter selected from the group consisting of the promoter of sequence of SEQ. ID NO: 9 and the promoter sequence of SEQ. ID NO:14.

48. The isolated nucleotide sequence of claim 47, wherein the sequence is SEQ. ID NO: 9.

49. The isolated nucleotide sequence of claim 47, wherein the sequence is SEQ. ID NO: 14.


SEQUENCE LISTING (29 sequences)

SEQ ID NO: 1 - DNA, 680 bp, Aspergillus niger
  CDS (1)...(678)
cataacgcggaatttcagggtctctggcccctccggacccagcaagaaaagaaggaagtctgttcggtcttcaacctccccgtcgagacggctaccaaatattgtcaattcggtaatgtgttcaacttgcttcacacaggagcgagttaccttcggttccatcagcgcgggtttggcgcggtcggtgtgtcaaagaagtacggaaagcgttcctgggctcggtatcctattttctggagtttggacaagatcggtagtctgcccaaccctgacccaactgatacggcggccctagaggatactcctgacgagaaagctctgactcggtcatatgaggaacggatcactgacaagctcgaggcccagaagtgggctggcctgaccgaggatcgcaacgccgacctcctggtgttcgtcggtcgctggtctaagcagaagggtgtcgatctgattgcggatgtcatccccgccgttctgtctgaccggccgcaggtgcaggtgatctgtgttggtcccatcatcgatctttacggaagactggctgccattaagctggggaggatcgctgccatgttccctggccgggtcttctcccgacctgagttcactgctttgcccccgtgtgtcttctctggcgctgacttcgctctgatcccgtctcgtgatgaaccatttggcct

SEQ ID NO: 2 - protein, 226 aa, Aspergillus niger
HNAEFQGLWPLRTQQEKKEVCSVFNLPVETATKYCQFGNVFNLLHTGASYLRFHQRGFGAVGVSKKYGKRSWARYPIFWSLDKIGSLPNPDPTDTAALEDTPDEKALTRSYEERITDKLEAQKWAGLTEDRNADLLVFVGRWSKQKGVDLIADVIPAVLSDRPQVQVICVGPIIDLYGRLAAIKLGRIAAMFPGRVFSRPEFTALPPCVFSGADFALIPSRDEPFG

SEQ ID NO: 3 - DNA, 510 bp, Aspergillus niger
  CDS (1)...(510)
aacgattatcatggtgcggcagctccactctatctcctacctcgaacgattccttgttgtctgtctctgcataacgcggaatttcaaggcctgtggccaatgcgaaatccccagcaaatacaggaggtctgccgagtgttcaatctggatcaggcggtggtcaagaaatacgtccaattcggcgacgtgttcaatctccttcactgtgccgcgaactatctgaaaattcaccagaacggatttggagcagtgggtgtctcgaagaaatatggcaagcgatcgtatgccagatatcccattttctggggtctcaagagcgttggagccctgccaaatcccgacccgtcggacttggctgactgggacagcaatgcgagcaagcagcctgaggaggtcaccgtcgacctagagttcgaggccagccgtggtgcactgaaaaaacaagcacaagaatgggcagggctgaaagttgaccccgacgctcagttgtttgtctttgtaggaagctgg

SEQ ID NO: 4 - protein, 170 aa, Aspergillus niger
NDYHGAAAPLYLLPRTIPCCLSLHNAEFQGLWPMRNPQQIQEVCRVFNLDQAVVKKYVQFGDVFNLLHCAANYLKIHQNGFGAVGVSKKYGKRSYARYPIFWGLKSVGALPNPDPSDLADWDSNASKQPEEVTVDLEFEASRGALKKQAQEWAGLKVDPDAQLFVFVGSW

SEQ ID NO: 5 - DNA, 447 bp, Aspergillus niger
  CDS (1)...(420)
acaaatgcggagttccagggtctttggcctgtccggacagccaccgaacgtgaagaggtacagtcagttttcaacctcagtgcagacgttatgaagcagtatgtgcaatttggtgaggtatttaacctattgcatgccggagccagttatctacgcatccaccagcgtggctttggcacggtgggtgtgtcgaaaaagtacggcaaacgatcctacgcccggtaccccatcttttggggattgcaaaaagtgggcaaccttcccaatcctgacccctctgacttgggagaatggagcaaggagcaagccatggcagtccaacgaggcgacgtcgcggtcgatccggattatgaggcaggtcgtggtgcgcttaaacttcaggcgcaagagtgggctgggctcaaccaggaccctgacgctgagctgttcgtatttgttgggcggtgg

SEQ ID NO: 6 - protein, 140 aa, Aspergillus niger
TNAEFQGLWPVRTATEREEVQSVFNLSADVMKQYVQFGEVFNLLHAGASYLRIHQRGFGTVGVSKKYGKRSYARYPIFWGLQKVGNLPNPDPSDLGEWSKEQAMAVQRGDVAVDPDYEAGRGALKLQAQEWAGLNQDPDA

SEQ ID NO: 7 - DNA, 438 bp, Aspergillus niger
  CDS (1)...(428)
cacaatgccgagtttcaggggctttggcctatgcgtacccagaaggagagggatgaggtctgttcagtcttcaatttagatactgacactgcaaggcgctatgtccaatttggagaagtcttcaatatgcttcatgccggagcaagctacctccgtatccatcaacaaggattcggtgcagttggtgtctctaggaaatacggcaaacgctcgtacgcacgctatcctatcttttggggcctgaagaaggtaggcaacttgcctaaccctgacccatctgatactgcagaatggaataaggaattgccgaaagacagtgagatcgaggttgatcccgaatacgaggctagcagggcaaacttgaagcgacaagcgcaacaatgggctggcttggagcagaatcccaatgcagatcttttggtgttcgtgggcagctgg

SEQ ID NO: 8 - protein, 142 aa, Aspergillus niger
HNAEFQGLWPMRTQKERDEVCSVFNLDTDTARRYVQFGEVFNMLHAGASYLRIHQQGFGAVGVSRKYGKRSYARYPIFWGLKKVGNLPNPDPSDTAEWNKELPKDSEIEVDPEYEASRANLKRQAQQWAGLEQNPNADLLVF

SEQ ID NO: 9 - DNA, 10173 bp, Aspergillus niger
  CDS (2781)...(3143)
  CDS (3207)...(3383)
  CDS (3440)...(10045)
  CDS (10101)...(10148)
gggcatcatccttcccaagcatggattcggggctgtgcggatgagtgagcatatcgccaagactgactgctaggaccatgatgagacggttcttccaacatcaaaatgacactagccaggaacccggtttggccccgatgaatatcaggtttctggcattggtttcgacccctgccgagattagatgtccctattcctccagggacttccaactctttctgtccccgatggatggaatatccaagcccccagctgcgtataatccgaccgtacactcacggagaaatccgaagccgataagatgtcccagcgatgttgtcagtttaggcgactggatgaaaaaatctgcgcatgcaagcagcagccaggaactgaattttttcttccgcagtgcacatgtggcattggggattttcagcctgcaggtacgacgtaccacgtacttacgtataggattgcctgcttattttctcctgttatttcccccttccagttgctttgtttttgtcccccaacaccaatgtacctgctgcatatgcagtacaacacagcagtcgttgaccgcagaagttccttagggtggtagatagtctgctgcaatgtcttggttttttttggggccaacgcgccatggcgggatggaagacgtgtggaaaccaacaagttgatctatggcagacattctatgtctgaacggcagcggtgaatgttagcgtctgattcatgtctgcgaccaagtgaagcagtcgactgcttctgcctacgtaatggcggcggttagtgttctatatatagcgctctgccgcgggaggacatattgcttgtgctcactttcgcccggtgagacaaacaactaatggtgtttggtgatctcaggctgtgaatagctaccagcgttgccggaattaactcgcatgaactacagagtcattcaacttcgcttcggcggcaatattattaaggattgtggtgaaatttggaagaacctctcccatcgtagtaagggttgtgatgatcctggtgggctttgatcgggtcacactgcccgtcccgcactgagatctagcatttctcttcgaccaacgacatcaccgcgagatcgctgatcttcaggaaggccaccccgtcacgtacgcgtccaaatcgctaacaagactgactgacgctctggctgaggtttgcctggcaatgtgcggcatgtcagcacgatgcacacgttgagggatccagatgcgaccgtggctcataggtgggatggtggcctagacagttttgcaacaagctttctgtgtgaaagaatcgaagaatcgcgaagcatttctgaggccgggctggagcctggacctggaaccacaaaaactcaatcggaatattgtctggtaggggccaagggcagaaggatggtctggcactcattctgtgacccggattgtgtcagtgtctggtggattatgtgcctggttgttgcccctccttcaacgacggggacatcccgaagcagaccgaaagactctgataggttccatgacgtcttacgctgtatgcgatttatgaggcgtttcttgggctgataacggccagactggccgcccagttagaaaccctcgacacagtgtagtccgacatcccgacggggccagtggcaatgcagccgaaccggggtcggcaggacaaggcatgacgccgtgatatgcctattggccaggtgtgaaggcatggggcagggtctctggcctctcactgtgaattatccgctgagataacagaataacaggatctggcgactcgctgaagggtacgtgggccacctggagtggggaggggatccgggtacatgcccactcacagtgattcaaagcaaagtcccgatggtttcggatctccaccgagccgcgcctggttccaaccatagaatctgcgcgaaggcaaaaaaaggaaaaaaagaaaagaaaagaaagaaatccctggtcgtgccgaaacacaaggaccaatgttcgccccatttgctggcccgacatcagatttttttggtaccttggcagtagttgatgggaacccacggatgtaccgttataggtcagacaggcacaggcacacaggaacagaaactctctcattctcattgagcttagaaatccatcctaggatgatcagattagcgctctgagtgtcggtcacagtgatcatctctcagtgactgcagtagtggcggctgcttcagcggtaaagtccgccagggatcaccaccatcagcatacacagctattataccctccacatccagcgcggttgcccattatcgaatgacagagggtgtactgacgaggcccgcctccacctcacccaatcatgctatcctgatatgacgtttcctaataatagccaccgagagtagagaatgaccgaacaggtgtcttggatcgcagggcctagcgaccccaccgcgggcgaaaggttttagataatggtctcacgacggcttaagcaccgcgcttgtctctctctcgtcatcgagtagtctcttgtctttcgtagcattcgcagcagtaactcccagcagcaatttgctgtcttcttttctctcgtttcaaccatcccctgggaacgctttcctcccacgtttctctcgtttcataccccaaagtcattcgttttttgtgctgcttatcttcgctgtgttcggtacgagggtttcgggcatgtggcattgggttcctgcataataatcctttggaccttggctggcacagaattcctggtaccacacgccgcttgccatcatgttcggcacgacggttcagcgctgcgtggtgctcattctggggctactcagcgtcacgaccgtgggctggccctacgatgagtcgctcgttgattacaacctgaacgagaacaagaccgcagagggaccgataaactattggggagaatggccagatcacacctatcatccgtcaccagacaactggcgcttccccatctataccatcttcctggatcgcatcgccaacggagatcccgctaatgacgatattaacggcaccacctacgagcatgtactcgattcgaatcagatgcgccatggcggtgacctagtcggcttgattgacacgctcgattacatcagaggcatgggcttcaaggtgagtctattccattgatatcagttcccggtccatccttgactgactgactgatcattctagggaatctacttcgctggtacgggcttgatgaatcttccctggggctacgatggttactccccggtggataccaccctcctcgacaagcaccatggcaccctgtccgactggaggcggaccatcaaggagatccacgaccgggatatgtatgtgatcatggataatacattagcaacgtgagtatgcaccgagtattctagacgaccatgccactaacaatctccccgtgcagtttgagtaacctgatcggtttcaagggccacttgaacgattcggctgatttcaatgcgaaagagtacgaggtggagtacgtcaccgagagaacgtacgccgatttccagttcagcaatgactacaatgacacctgcaactacccgaaattctggaacgagacgggatacccgttgacgtccggtggcgtgcaagacctgaagggttgctacaacagtgatttcgatcagtatggcgaactcgaggcgtttggtaatttccccgactggaagcgacagctcacgaaattcgcttcggtgcaagatcgtctgcgagaatggcacaagccggttcgcgatgtcatcacccggcattcctgcattgtgatcgccagtttggacattgatggcttccggtttgataaggccgtccaggccactctggatccgctgggtgacatgctagcggtctaccgtgaatgtgccaagcaatatggcaagaagaacttcttcctgccgggagagatcacttcgggaaacacctttggtagtctctatcttggccggggtcgtcagccaaaccagacgcccgattcggcagacgctggcgcgaagttgacgaatgcttcgtcggattcctacttcctccgcgatgatggcctgcaggcgttggacgctgcagcctttcactacaccatctatcgttcgatgacccgtttcctgggaatggacgggaacctggtggccggctttgacctgcccactgatttcatcgaagcctggaatggcatgctcgtcactaacgacttcatcaatgccttcacgggcgagttggatcccagacatatgtatggagtgtcgaaccaggataactttcgctggcccgccatcaagaatggtacggagaaatacctgctgggtctatacattgttacgctggagctgccgggaatccctttggttctctggggcgaggagcaggagatgtacgtgttcgatgcgactgcgtccaactatctgttcggtcggcaacccatgacttaccagacggcgtggtggacgcatggctgcttctcgctaaacacctcgaaattctacgatttccccaacgacaagggattgaatggttgcaacgatatcacagtgacttacgaccaacggaatccggcgcacccccttcgcaacatcatgaagcgcatgtttgagattcgggagcagtacccagtggccaatgatggcttcaaccttgagaccatttcccagcttacccaggatatctatcttccgggttcatcgactaccccgacggtgactggtctctggtcagtgctgcgcagctactaccccggggttcagaaagaggcctcgagtagcaacgatactctttggcttgtatatcataatgccaataaaacggaaacctatggcaagaactgtactagcaaggactccgctcttctgtccccttacgagtcgggcacgaagttgaaaaacctcttctatccctacgatgaactcacgctgcaggatggaccaagtgacgctagcaacggcaccgagtcctacgggtgcacgaccaacatgaaattgctgccatgggaattccgggcttacgtcaaggcgagcgacttcgttgaacctggccctaccgtcaccgagtttgtccccggccacgatgctcgcctgctgtcttccgaggatactggggaaacactgaagatccagctgggctactccaaggccatggactgcagtgccatcacgaaagcagtctccctgaattcgaccaccgtcaagggcgtcaatgccacccttgacacctctagcgtgagctgcaccaacgtcaccgccaggacgagcagtaataactacatcggcgaggttcccactgtttggacctggtccgcgaacctgacaaacgtctatcacggaatccaccagctcaccgtcaagaatgcctccacgacgtctggaacgcgcgtagatgccatcgaccgctttttattccgagtcggaacccacaccaacccgttaatatcgccactggccaactactctaccagcctcgtccatcagtcggataacggcagctactatgttcagcatgatgcggccggcgctgaccagttccgctacacgaccgactttggcctgaactggtccaactggacagactacactggtggtaacacgacgattgaatttccgacttggaccggcacagatgctcaaaagtgggagggcactcacattcgcgtccagtatttctcccgccttaccggcagcagtgattacatccaggagggtgattatgactgggaatccggtgtccctcgcagatttccccacctgtggtggaatggcccgtacaaccaatacggttatgatgccggtctggacagcaagatgcggtttgatactaaggatcgccgctggaaatatgatttcgtctatgaatggccgtccgtcggacagatcagtgtctggggcactggcaaggacggtgttccggagacaacgaatgtttatggcgatgttgacaactcatcggttgtacaggtcttgcctccctcctacttgtcgtcgaacgtgattaatatcaccgagctgccccccttcccgcaccttggctggaccatctctctcaatgatgccaacctccgctatgaattgcttccggtcggatctggatgggcccagctggtactttacatccttctctgggtcgtccctatcttgatggggctggcgggagttttcatcttcatccgcaccttctaccgtgttgagctgaataccgatggcaatgttgttaaggaggacaagctgccgctgttgttctggcgcaggatgaagggccaatctggaggtgatggccagatggaccgggaagaatcagatgtcgccgccgtggcaggcgaaatggccattgcaggagcccccgagaaacgccgcacggtcttgatcgccaccatggagtacgatattgcggattggcaggccaaggtcaagattggtggtctgggagtcatggcccagctcatgtcccaaaacctgggatatcagaatcttatctgggtggttccctgcgtgggagacatcgaatatcccgaggataccccgacagagccatacatcgtgaagatcctcgacaacccctatctcgtcaatgtgcagtatcacgttctcaacaacatcacctacgtccttcttgacgctcctgtgttccgccagcagacaaaagccgagccgtatcctccccgcatggatgacctggatagcgccatttactactccgcctggaaccaatgtatcgcagagaccatcaagcgcttcccgtccatcgatctgtatcacatcaacgatttccacggatgcctggcaccgttgtacctcctgcccacacgcacgatccccgtgtgtttgtctctccacaacgccgagttccagggtctctggcccctccggacccagcaagaaaagaaggaagtctgttcggtcttcaacctccccgtcgagacggctaccaaatattgtcaattcggtaatgtgttcaacttgcttcacacaggagcgagttaccttcggttccatcagcgcgggtttggcgcggtcggtgtgtcaaagaagtacggaaagcgttcctgggctcggtatcctattttctggagtttggacaagatcggtagtctgcccaaccctgacccaactgatacggcggccctggaggatactcctgacgagaaagctctgactcggtcatatgaggaacggatcactgacaagctcgaggcccagaagtgggctggcctgaccgaggatcgcaacgccgacctcctggtgttcgtcggtcgctggtctaagcagaagggtgtcgatctgattgcggatgtcatccccgccgttctgtctgaccggccgcaggtgcaggtgatctgtgttggtcccatcatcgatctttacggaagactggctgccattaagctggggaagatcgctgccatgttccctggccgggtcttctcccgacctgagttcactgctttgcccccgtgtgtcttctctggcgctgacttcgctctgatcccgtctcgtgatgagccatttggattgattgccgttgagttcggtcggaagggtgcactgggaatcggatctcgcatcggaggtttgggtcagatgccaggctggtggtacaccgttgaatcagacgccactcgccatcttctacaccagctgaagactgcgatcaaatctgccctggattcgactcctgaaactcgagcccagatgcgcgcaaactccgctaagcagcgattccccgttctcgagtggatccagaaactcgagactttgcagcggacatcgatcaagattcatcatgacaagaacaaggacaccgtgactggttcgatcccggagtctgagagcgtatgggacctgcaaaaccttcagagtgcgcgctattccagtgtggcacttcctaatatggctctgtccagagcggacggcacggaaaccccgcctgaagccatgatccagcaggcccacgctcgccttcaagagatccaggcatccgagggaagcagtcgcgacagcagcctcaaccgcaaactgtcactgggtcgccggtccggccctggtcagggcagaaagcgcttgacgaagaggcaagcgaccgcaagccaggcatccactgatcacggagaagatgagaacgataccgacgctgacgacgaccacaccaggccgcaggaggattacatttctcccgaggaggccatgcaggccgttaacaacactctaactccccatgctatcactggagacagtactccccgcgcctcctacttgtctagacccgtatccccgtaccccatctcacagatctctactccagcgtgcccgacaccgccacccatgactcacttccggaccgtttctatgctgtcgttgccatccgttgttggggaccataaccgccttagcagtctcggcggcgaggagcagaaccagcccgtcttcgagttgcagaaggtggacccgactttcacggacagcttgggccacttcacccggcgcttcgagcagtcgcttgagaagttgaacaagaagaactccatcacggactattgcattgaggtctatctgatgaagagtgagcgcaagttctacaattcatacaacgacgcccagcttaagaagcagcctaagaaccgttcgctcgcggttcccagctctgagagtgttgtgcaagaagggcccggtggctcagtcgttcgtcactcgtactccagtgaggattctgaaatcaatggggaggatgagattgaccgctggctgatgcggttgggttacacgcgtccgatcgcagttcagcgattcatgagacgccgcttgggcaattggccagtgtattcactattcctgggtctgggtcagatcatcgccaccaactccgcacaaatcaccctgcttgtcggccaggtcggagagaccgctgtgaagctttatgtcatcgccgctatttactgcctgtcctccatcgcgtggtggttcctgtatcatcggttcccagccgtcatctccctcagtcttccctggttcatctactgcatggcattcatcgttatcggtgtttcacccttcggtctcacggctctcggtcgggcctgggctcagaatgttgcggctggcgtctacgccattgcgtcgtctagcggttccttgttcttcgctctgaactttggagaccagggtgccgtccctgtcaaggactggatgttccgcgccagtatcattcaaggtatctcacagatctacacggtcgcgctctggttctggagctccaaggtcactgccgccgaagtgggaggtgtatctgtagcggccttgagctcctggcgtctcacggccgtggtgatgcccattgctgccctgtgcttcgtgatcggagtcctgctggccctgggattgcccaactactaccgtcaagctcccagccgtatcctgttcttctacaagtctctgttccgccgacgcatcatcctgtggttcttcttcatggtcatcgtccagaactggttccttgctgccgctttcggccgcaactggtccttcctctggtcgtccaaacacaccaagccctgggagattgtcctccttgtgttcggcttcttcgtcgtcctatgggtagtcattcttgtcatattccgtgccctgtcgaaggaacacagctggatcttgccagttttcggtctgagtctgggtgctccccggtgggcacaaacctggtggggaacctccaacatcggctactatctcccttgggctggcagcttggtgtcggggtcgatcgcttcccgctgcctgtggctctggctgggactccttgacgagatccaacaagtcggattgggtatgattctgctgcagaccatgactcgcgtgcatgtctgcttcgtcctgttggccgcgcaagcactgggatccatcgccaccatctgcgcccgcggcttcgcgccgaacaagattggccctggggatatctctccggatgtgggcacgtcggtggacaaggtcggtaacgcatggttctggatcgctctcttcttccagctcctggcaaggtaagccttcccacactttcttttctcacgcgagctttatctaacgtttgaacagttggggcttcctgctcttctaccgccgcgaacagctcaaccgaccctaaatgccctactgggtcaatacagatc

SEQ ID NO: 10 - protein, 121 aa, Aspergillus niger
MFGTTVQRCVVLILGLLSVTTVGWPYDESLVDYNLNENKTAEGPINYWGEWPDHTYHPSPDNWRFPIYTIFLDRIANGDPANDDINGTTYEHVLDSNQMRHGGDLVGLIDTLDYIRGMGFK

SEQ ID NO: 11 - protein, 59 aa, Aspergillus niger
GIYFAGTGLMNLPWGYDGYSPVDTTLLDKHHGTLSDWRRTIKEIHDRDMYVIMDNTLAT

SEQ ID NO: 12 - protein, 2202 aa, Aspergillus niger
LSNLIGFKGHLNDSADFNAKEYEVEYVTERTYADFQFSNDYNDTCNYPKFWNETGYPLTSGGVQDLKGCYNSDFDQYGELEAFGNFPDWKRQLTKFASVQDRLREWHKPVRDVITRHSCIVIASLDIDGFRFDKAVQATLDPLGDMLAVYRECAKQYGKKNFFLPGEITSGNTFGSLYLGRGRQPNQTPDSADAGAKLTNASSDSYFLRDDGLQALDAAAFHYTIYRSMTRFLGMDGNLVAGFDLPTDFIEAWNGMLVTNDFINAFTGELDPRHMYGVSNQDNFRWPAIKNGTEKYLLGLYIVTLELPGIPLVLWGEEQEMYVFDATASNYLFGRQPMTYQTAWWTHGCFSLNTSKFYDFPNDKGLNGCNDITVTYDQRNPAHPLRNIMKRMFEIREQYPVANDGFNLETISQLTQDIYLPGSSTTPTVTGLWSVLRSYYPGVQKEASSSNDTLWLVYHNANKTETYGKNCTSKDSALLSPYESGTKLKNLFYPYDELTLQDGPSDASNGTESYGCTTNMKLLPWEFRAYVKASDFVEPGPTVTEFVPGHDARLLSSEDTGETLKIQLGYSKAMDCSAITKAVSLNSTTVKGVNATLDTSSVSCTNVTARTSSNNYIGEVPTVWTWSANLTNVYHGIHQLTVKNASTTSGTRVDAIDRFLFRVGTHTNPLISPLANYSTSLVHQSDNGSYYVQHDAAGADQFRYTTDFGLNWSNWTDYTGGNTTIEFPTWTGTDAQKWEGTHIRVQYFSRLTGSSDYIQEGDYDWESGVPRRFPHLWWNGPYNQYGYDAGLDSKMRFDTKDRRWKYDFVYEWPSVGQISVWGTGKDGVPETTNVYGDVDNSSVVQVLPPSYLSSNVINITELPPFPHLGWTISLNDANLRYELLPVGSGWAQLVLYILLWVVPILMGLAGVFIFIRTFYRVELNTDGNVVKEDKLPLLFWRRMKGQSGGDGQMDREESDVAAVAGEMAIAGAPEKRRTVLIATMEYDIADWQAKVKIGGLGVMAQLMSQNLGYQNLIWVVPCVGDIEYPEDTPTEPYIVKILDNPYLVNVQYHVLNNITYVLLDAPVFRQQTKAEPYPPRMDDLDSAIYYSAWNQCIAETIKRFPSIDLYHINDFHGCLAPLYLLPTRTIPVCLSLHNAEFQGLWPLRTQQEKKEVCSVFNLPVETATKYCQFGNVFNLLHTGASYLRFHQRGFGAVGVSKKYGKRSWARYPIFWSLDKIGSLPNPDPTDTAALEDTPDEKALTRSYEERITDKLEAQKWAGLTEDRNADLLVFVGRWSKQKGVDLIADVIPAVLSDRPQVQVICVGPIIDLYGRLAAIKLGKIAAMFPGRVFSRPEFTALPPCVFSGADFALIPSRDEPFGLIAVEFGRKGALGIGSRIGGLGQMPGWWYTVESDATRHLLHQLKTAIKSALDSTPETRAQMRANSAKQRFPVLEWIQKLETLQRTSIKIHHDKNKDTVTGSIPESESVWDLQNLQSARYSSVALPNMALSRADGTETPPEAMIQQAHARLQEIQASEGSSRDSSLNRKLSLGRRSGPGQGRKRLTKRQATASQASTDHGEDENDTDADDDHTRPQEDYISPEEAMQAVNNTLTPHAITGDSTPRASYLSRPVSPYPISQISTPACPTPPPMTHFRTVSMLSLPSVVGDHNRLSSLGGEEQNQPVFELQKVDPTFTDSLGHFTRRFEQSLEKLNKKNSITDYCIEVYLMKSERKFYNSYNDAQLKKQPKNRSLAVPSSESVVQEGPGGSVVRHSYSSEDSEINGEDEIDRWLMRLGYTRPIAVQRFMRRRLGNWPVYSLFLGLGQIIATNSAQITLLVGQVGETAVKLYVIAAIYCLSSIAWWFLYHRFPAVISLSLPWFIYCMAFIVIGVSPFGLTALGRAWAQNVAAGVYAIASSSGSLFFALNFGDQGAVPVKDWMFRASIIQGISQIYTVALWFWSSKVTAAEVGGVSVAALSSWRLTAVVMPIAALCFVIGVLLALGLPNYYRQAPSRILFFYKSLFRRRIILWFFFMVIVQNWFLAAAFGRNWSFLWSSKHTKPWEIVLLVFGFFVVLWVVILVIFRALSKEHSWILPVFGLSLGAPRWAQTWWGTSNIGYYLPWAGSLVSGSIASRCLWLWLGLLDEIQQVGLGMILLQTMTRVHVCFVLLAAQALGSIATICARGFAPNKIGPGDISPDVGTSVDKVGNAWFWIALFFQLLAR

SEQ ID NO: 13 - protein, 15 aa, Aspergillus niger
WGFLLFYRREQLNRP

SEQ ID NO: 14 - DNA, 5867 bp, Aspergillus niger
  CDS (1072)...(1077)
  CDS (1136)...(1204)
  CDS (1296)...(1529)
  CDS (1601)...(2992)
  CDS (3046)...(3207)
  CDS (3262)...(3480)
  exon (1072)...(1077)
  intron (1077)...(1134)
  intron (1206)...(1296)
  intron (1529)...(1599)
  intron (2994)...(3046)
  intron (3209)...(3262)
tacgcacggtggcccctttaaaacacatcgcgacaaatacccctccccctttcccactcaattatctcggttcccatgggcaagttcacaccagaggcaaaatctagtgtgggatgcgactcaagaacaaagaggggcaccctcacgatttagggcctctatctgctaggttctttgtcctaagacacatactctgtacataactactaccgacatgggtgctgcacctgcttttacctgtcgtcaataacttgatgtgttgcatgttccaactgtagtagtaacatgtctttcaagcaggtcaccactaccatcgatagggagggcgaagtgatagtatatttattacctagtactgaagttattattagaaacctatgtttagaagtcccctagatcatatactccgtctcagcattagaaccagatccatggcgtcagggaaagaatttgcctaacttgactaataatagttaaaagtggagcgctattctgacgatgcaggtgggggtgagtattctacttcggtatctactgagcaattcgtacaagctagtacaataatactcaatcgctaccttggatgcggatggtcctggcctgactcaggtcacgggtgaaacataccagtcgattgtgtgaggcgcagggtgggcttattggctgctacctcatttgacgaatggctgagtgtgcctatagcagcttccctggtctgtgtgccaaggtgggtcgatctcaccacataaaacataacagctccccctcctttctaccctcccaattgtttgttgatcccagtgaaccacctcctcaacctctccgtgtcctttcctcctgattcctccttctttcaaatccccctttcatacctacacacttgttcatcactccttttcataagtagtaagttacatacattgccgaatgcaatgttgtcggtgtcttgattaacattgttctcttcaggtgaactcctttcgatatctagacatagtgtcgtcttcttggaacatcaaacggtcccctagtctctcccacgcctccgagtaatcacatcaaaaaataatcatgtggtatgtatggctccaaagctcaatttgtatattgcgatgtctaattcaaactttaaagtggcattttcggctacatcaactaccttgtcgaaagagaccgcaggttcattctcgatactatacttaacggtcagtccctctccatcgtgccagcctccatcaggtctcacatcgctccgcgaacatgtcctattacatcttgtatcctgacatcgcgtaggtctttctagactcgaataccgcggttatgattcagccggtctcgcagtcgatggcgacaagaagaaggaagtctgtgccttcaaggaagttggcaaggtcgctaagttgaaggaactcattgaagagtccaagcccgatttcaccaagacttttgagtcccatgctggcattgctcacacacgctgggctactcatggtactccgtctcgccagaactgtcatcctcacaggtgggttcactgaatttacgtcgattggattaggcttaggaaacatgagactaacacatgccaccttccagatctgatgccaactgggagtttgctgttgttcacaatggtatcatcaccaactacaaagaattgaaagccttgctggagagcaaaggattccgcttcgagaccgagactgacacggaatgcatcgccaagcttactaagtatctttatgatcagcagccagacattgacttcacggtgttggccaaggctgttgtcaaagaactcgaaggcgctttcggtctcctcatcaagtctgtgcactatcctcatgaagttattgcggcccgcaaaggttccccgcttgtcattggtgtgcgaacttcaaggaagatgaaggtggacttcgttgatgtggaatactccgaagatggccccctcccagcggagcaggcttctcaaaacgttgccatcaagaagtctgctactggattgcttgcacctcctgacaagtcactcctgcaccggtcccagtctcgcgctttcctttccgatgacggcgttcctcagccagccgaattcttcttgtcatcagacccatcagcgatcgttgagcacactaagaaggtcctttacctagaagatgatgacattgctcatattcatgagggacagctgaacatccatcgtttgactaaggatgatggcacgtctaatgtccgcgccattcagaccattgaacttgagctacaggagattatgaagggtaactttgatcactttatgcagaaagagatctttgagcagcccgaatccgtggttaacactatgcgcggtcgactggatgtcgcaaacaagcaggtcacactgggcggtctccggcagtatatttctaccatccgtcggtgcagaagaatcatcttcgtagcttgtggaaccagttaccactcatgcatggctgtgcgtggtgtttttgaggagcttactgagattcctatctcagttgaacttgcctcggacttcctggacagacaagcgcctgtgttccgtgacgacacatgtgtcttcgtttcccagtcaggagagacagccgattccctgatggccttgcgctattgtctcgagcgtggtgccttgactgttggtattgttaatgttgtcggctcatccatctccctcctgactcactgcggtgtgcacatcaatgctggtccagagattggtgtcgcttctacgaaggcatacacatcgcagttcgtggccatggttatgtttgcactctcgcttagcgaggatcgggcttccaagcagaagagacgcgaggagatcatggagggtttggccaaggtatcagagcaattcaaggagatcctgaagctcaatgagcccattaagcagatgtgcgccaagttcttcaagaaccagaagagtttgctgctgttgggtagaggtggccagttccctacggcccttgaaggttagcaggttgatctttcacataatcactcctcagttaacatgattttataggtgcgctcaagatcaaggagatctcctacctgcactgcgaggctgttatgtcaggcgaactgaaacacggtgtccttgcccttgtcgatgagaacctacccatcatcatgatccttacgagggataacatcttctcaaaatcactgaacgcttatcagcaaggctagtcaatcctccgctccaatccgtaagctttagtcactaaccatcctacagtcattgcccgaggaggccgccctatcgtgatctgcaattacgatgatccagagttctctgccgctcaaacagaaaagattgaggtcccgaagacggtcgattgtcttcaaggtcttttgaacgttattccattacagcttatttcctactggcttgcagtgggagagggtctcaacgtcgatttcccccgtaacctcgcgaagtcggttactgttgaatagagcgacgaagcaaaaacgatagattcgtgggcacgagactgggaattctcaattaatccatagggtctctaaattaatgatgttaacatataaatcctgcagtattggtagtttttacccatgattctcctagttgcggtttgcttctgcctccctgctgttgtaatcgtctgtctgaggtgtgtccgacgttttgtcgcatggcaaaccgtagcagccagtagtctccagcgatttagccttgcacatatcgagtcgaatgcaataacgtggtcttccatgggttaagagtgagggacctggggacatcttattacatcagtaagtactgctaactagataactagctcaaaagatcattatccttgatccagcggggtagacttccgttccccactttgcgccggaactcccacttctgttgcacattgtttgtccgtctcatcagtttccccgaacaccatcttccttcaatttgacaacggtagcctattaggaccaatatctggaaatttcattacaggattcggacgtgatgacttctgtgtctctgaaaggcccaggcgctgcgcagacatacgatggtaataacctctttcaggatgatcgtatgattaatgatccgtctgctaaatgatggaatcttaggggctgggctacgaatcgccattgtccatgcgaggtggaatatgggcataattggtcctctcgtggatggggctaagcggagcctccttgcagctggagtttctgagaataacataaccgtccacactgttccaggcagttatgagttgcccttcgccgtgcagaagtaagcatattgtcatcatgtcttttccttctcgggcatttcattggagacttcggagactgatgctgactttgtacgctgtcaggttatacgcggcttcccagattgaagcttccaaaggctcagcctcggcgcaaggcatcagtgccaccgatttgctgtcctcctctacagcggacctcacgaaggcatctccacagtcgccatcttccgatacgcctagaccttttgatgccatcattgccgttggagtgctgattaaaggggagactatgcatttcgaatacatcgcggatgccgtctctcatggattgatgcgtgtgcagctcgatacgggagtgccggtcatctttggcgtgcttactgtcttgactgaagagcaaggcttggaaagagccggcatgggcaaaaaagggatgcacaaccatggagaggactggggtaatgctgcagtagaacttggagcaaagagaagagactgggctgagggtaaagttgtttagtggaccttcggcaaggcatcgatttcttttccgttttgagttggttcgaagttgatttacggtaatcgatcacctctacaaaagaagaataatcgattgctttacgttgaagatcggaaccttgattggcggccttaagtcacagttgccatatagcatacattcgtttaccgtgcattaagatatgaaggtcacatgaataagggcaaaataacgagatggattattctttgaccgagttccaagtgtagggttaaggctataaagttcactcctccatccgttcattctcgttcgagtcacctctttgttgcgtcttcgtcctcatcctctccctccagagctctctttgctgcccgctctgcctcctcacggagttctctaggtacctcctcgtgcctcccctttgtcggaccgtctaatgaactgatccaggtcatttccagttcaaaatccttgtccttactgtcttcatgggcgacataaataattcgagccgcttccttcactccatcgagaaggctcagtgtgccggagctcaggtcgagtttctcaagctcagccttggctgcttgtctccctttcccggtggcagcgccataataacccttcgatagcgtcagcatcgtctaaaagcacctttgtagattgcaagaacatacccagtacaggccgcttggttcaatcatatatagacctgggcccccagtcttagcgccctcgaccttaccaccggaacccgccttgggcccacttccgacttgaccgtcgacagcaagctcggcttcggagtcccagcctccgacaatagcggtcacgccaaaaggcctgacactagagtagagtgtgtaggcttgcacgtaactcccaaggcgattggatagcgcggaggttggaattggccctttgtagacacttctccacgatgaagcctcgtcacgggcccgagaaacgaaatgtcgcccgtctggcaccagtccggcggaaacctgtcatggaagcaacatattagcgcgacaggcccctaaaaagcgtagatgcccactatatggaatcagcgtggatcc

SEQ ID NO: 15 - protein, 23 aa, Aspergillus niger
GIFGYINYLVERDRRFILDTILN

SEQ ID NO: 16 - protein, 78 aa, Aspergillus niger
GLSRLEYRGYDSAGLAVDGDKKKEVCAFKEVGKVAKLKELIEESKPDFTKTFESHAGIAHTRWATHGTPSRQNCHPHR

SEQ ID NO: 17 - protein, 464 aa, Aspergillus niger
SDANWEFAVVHNGIITNYKELKALLESKGFRFETETDTECIAKLTKYLYDQQPDIDFTVLAKAVVKELEGAFGLLIKSVHYPHEVIAARKGSPLVIGVRTSRKMKVDFVDVEYSEDGPLPAEQASQNVAIKKSATGLLAPPDKSLLHRSQSRAFLSDDGVPQPAEFFLSSDPSAIVEHTKKVLYLEDDDIAHIHEGQLNIHRLTKDDGTSNVRAIQTIELELQEIMKGNFDHFMQKEIFEQPESVVNTMRGRLDVANKQVTLGGLRQYISTIRRCRRIIFVACGTSYHSCMAVRGVFEELTEIPISVELASDFLDRQAPVFRDDTCVFVSQSGETADSLMALRYCLERGALTVGIVNVVGSSISLLTHCGVHINAGPEIGVASTKAYTSQFVAMVMFALSLSEDRASKQKRREEIMEGLAKVSEQFKEILKLNEPIKQMCAKFFKNQKSLLLLGRGGQFPTALE

SEQ ID NO: 18 - protein, 54 aa, Aspergillus niger
GALKIKEISYLHCEAVMSGELKHGVLALVDENLPIIMILTRDNIFSKSLNAYQQ

SEQ ID NO: 19 - protein, 73 aa, Aspergillus niger
VIARGGRPIVICNYDDPEFSAAQTEKIEVPKTVDCLQGLLNVIPLQLISYWLAVGEGLNVDFPRNLAKSVTVE

SEQ ID NO: 20 - DNA, 438 bp, Penicillium chrysogenum
  CDS (1)...(438)
cacaacgctgaatttcagggcctatggccgatgcgcacacaaaaggagaagaccgaggtgtgctccgtcttcaatctcgatatagaagttgtcgctcgctacgttcagtttggtgaggtctttaatttgcttcacgcgggtgcgagctacttgcgttttcatcagcaaggttttggcgctgtcggagtgtccaaaaaatatggcaagagatcttacgcacgctatcctatcttctggggcctaaagaaaattggtaatttaccaaaccctgatccctcggatactggagagtggaataaagagttacctaaagagagtgagatcagtgttgacagcgagtacgaagttcggcgggctgaattcaagcgtcaagcacaggaatgggctggtcttgaacaaattcccaatgccgatctaatggtattcgtaggccgttgg

SEQ ID NO: 21 - protein, 146 aa, Penicillium chrysogenum
HNAEFQGLWPMRTQKEKTEVCSVFNLDIEVVARYVQFGEVFNLLHAGASYLRFHQQGFGAVGVSKKYGKRSYARYPIFWGLKKIGNLPNPDPSDTGEWNKELPKESEISVDSEYEVRRAEFKRQAQEWAGLEQIPNADLMVFVGRW

SEQ ID NO: 22 - DNA, 432 bp, Penicillium chrysogenum
  CDS (1)...(432)
cacaacgcggagtttcagggtctttggcctatgcgtactcaaaaggagagacgcgaagtctgccaggttttcaacttggacgaggacgtcgctcgcaagtgtgtgcaattcggtgaagttttcaacctcctccacgccggtgctagctatcttcgggtcaaccaacagggattcggtgccgttggtgtctcaaagaagtatggtaaacgttcttatgctcgttacccaattttctggggtcttcgcaaggtcggcaatctgcccaacccggatccttctgatgttggagagtggaccaagcagcccgtcacggaggccaccgttgatcccgagtatgaggctggtcgtgccgagttgaagcgtcaggctcaagagtgggctggactcgagcagaacccggatgctgaccttctcgtgttcgtgggcagctgg

SEQ ID NO: 23 - protein, 144 aa, Penicillium chrysogenum
HNAEFQGLWPMRTQKERREVCQVFNLDEDVARKCVQFGEVFNLLHAGASYLRVNQQGFGAVGVSKKYGKRSYARYPIFWGLRKVGNLPNPDPSDVGEWTKQPVTEATVDPEYEAGRAELKRQAQEWAGLEQNPDADLLVFVGSW

SEQ ID NO: 24 - DNA, 441 bp, Penicillium chrysogenum
  CDS (1)...(441)
cataacgcggagtttcaaggactctggccgatgaaaaccacactgcaaaatcaagaggtttgcagtgtctacaaccttgaccaagcattggtgaagaagtatgtacaatttggtgaggtgtttaaccttcttcatggagctgccaactatctgaaaattcaccaagacggctttggtgctgtcggtgtctccaagaaatatggcaagagatcgtacgccagatatcccattttctggggtctcaagagcattggagcgctaccaaaccccgatccgtctgatactgccgagtggaatcacaacgcaaacaacaatctcgaagacgttgtcatcaatgaagaatttgaagccagtcgtgtcacactcaagagacaagcgcaagaatgggcaggccttgaagttgacccagaggcccagctgtttgtattcgtcggacgatgg

SEQ ID NO: 25 - protein, 147 aa, Penicillium chrysogenum
HNAEFQGLWPMKTTLQNQEVCSVYNLDQALVKKYVQFGEVFNLLHGAANYLKIHQDGFGAVGVSKKYGKRSYARYPIFWGLKSIGALPNPDPSDTAEWNHNANNNLEDVVINEEFEASRVTLKRQAQEWAGLEVDPEAQLFVFVGRW

SEQ ID NO: 26 - DNA, 182 bp, Fusarium oxysporum
  CDS (1)...(180)
gagtataggggttacgactctgctggtctagccatcgacggcgacaagaagaaggaggttctcgctttcaaggaggttggcaaggtcgccaagctccgaaagctcattgacgagtccgacctcgacctcgagaagatctttgattcacatgccggtattgcccacacccgattggccacgca

SEQ ID NO: 27 - protein, 60 aa, Fusarium oxysporum
EYRGYDSAGLAIDGDKKKEVLAFKEVGKVAKLRKLIDESDLDLEKIFDSHAGIAHTRLAT

SEQ ID NO: 28 - DNA, 182 bp, Penicillium chrysogenum
  CDS (1)...(180)
gaatacagtgggtacgattcggccggtatggctgtcgatggtgacaagaaaaatgaggtgcaggctttcaaggaagttggcaaggtcgctaagctccgtgagctcatcgccgagaccaagcccgacatgaccaagaccttcgagtcccacgctggtatttctcacacacgattggccacaca

SEQ ID NO: 29 - protein, 60 aa, Penicillium chrysogenum
EYSGYDSAGMAVDGDKKNEVQAFKEVGKVAKLRELIAETKPDMTKTFESHAGISHTRLAT